(19) United States
(12) Patent Application Publication
(10) Pub. No.: US 2010/0035967 A1
(43) Pub. Date: Feb. 11, 2010

(54) MODULATION OF TOLL-LIKE RECEPTOR 9 EXPRESSION BY ANTISENSE OLIGONUCLEOTIDES

(75) Inventors: Ekambar Kandimalla, Southboro, MA (US); Mallikarjuna Putta, Burlington, MA (US); Lakshmi Bhagat, Framingham, MA (US); Daqing Wang, Bedford, MA (US); Dong Yu, Westboro, MA (US); FuGang Zhu, Bedford, MA (US); Sudhir Agrawal, Shrewsbury, MA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Idera Pharmaceuticals, Inc.

(21) Appl. No.: 12/510,469

(22) Filed: Jul. 28, 2009

Related U.S. Application Data
(60) Provisional application No. 61/084,091, filed on Jul. 28, 2008.

(51) Int. Cl. A61K 31/7088; C07H 21/04; A61P 37/00; A61P 29/00

(52) U.S. Cl. 514/44 A; 536/23.1

(57) ABSTRACT

Antisense oligonucleotide compounds, compositions and methods are provided for down regulating the expression of TLR9. The compositions comprise antisense oligonucleotides targeted to nucleic acids encoding TLR9. The compositions may also comprise antisense oligonucleotides targeted to nucleic acids encoding TLR9 in combination with other therapeutic and/or prophylactic compounds and/or compositions. Methods of using these compounds and compositions for down-regulating TLR9 expression and for prevention or treatment of diseases wherein modulation of TLR9 expression would be beneficial are provided.

### RELATED APPLICATIONS

This application claims the benefit of prior U.S. Provisional Patent Application Ser. No. 61/084,091, filed on Jul. 28, 2008, the contents of which are incorporated by reference in its entirety.

### BACKGROUND OF THE INVENTION

1. Field of the Invention

The present invention relates to Toll-Like Receptor 9 (TLR9). In particular, the invention relates to antisense oligonucleotides that specifically hybridize with nucleic acids encoding TLR9, thus modulating TLR9 expression and activity, and their use in treating or preventing diseases associated with TLR9 or wherein modulation of TLR9 expression would be beneficial.

2. Summary of the Related Art

Toll-like receptors (TLRs) are present on many cells of the immune system and have been shown to be involved in the innate immune response (Hornung, V. et al., (2002) J. Immunol. 168:4531-4537). TLRs are a key means by which mammals recognize and mount an immune response to foreign molecules and also provide a means by which the innate and adaptive immune responses are linked (Akira, S. et al. (2001) Nature Immunol. 2:675-680; Medzhitov, R. (2001) Nature Rev. Immunol. 1:135-145). In vertebrates, this family consists of at least 11 proteins called TLR1 to TLR11, which are known to recognize pathogen associated molecular patterns (PAMP) from bacteria, fungi, parasites and viruses and induce an immune response mediated by a number of transcription factors.

Some TLRs are located on the cell surface to detect and initiate a response to extracellular pathogens and other TLRs are located inside the cell to detect and initiate a response to intracellular pathogens. Table 1 provides a representation of TLRs, the known agonists therefore and the cell types known to contain the TLR (Diebold, S. S. et al. (2004) Science 303:1529-1531; Liew, F. et al. (2005) Nature 5:446-458; Hemmi H et al. (2002) Nat Immunol 3:196-200; Jurk M et al., (2002) Nat Immunol 3:499; Lee J et al. (2003) Proc. Natl. Acad. Sci. USA 100:6646-6651); (Alexopoulou, L. (2001) Nature 413:732-738).

TABLE 1

| TLR Molecule | Agonist | Cell Types Containing Receptor |
|---|---|---|
| Cell Surface TLRs: | | |
| TLR2 | bacterial lipopeptides | Monocytes/macrophages |
| | | Myeloid dendritic cells |
| | | Mast cells |
| TLR4 | gram negative bacteria | Monocytes/macrophages |
| | | Myeloid dendritic cells |
| | | Mast cells |
| | | Intestinal epithelium |
| TLR5 | motile bacteria | Monocyte/macrophages |
| | | Dendritic cells |
| | | Intestinal epithelium |
| TLR6 | gram positive bacteria | Monocytes/macrophages |
| | | Mast cells |
| | | B lymphocytes |
| Endosomal TLRs: | | |
| TLR3 | double stranded RNA viruses | Dendritic cells |
| | | B lymphocytes |
| TLR7 | single stranded RNA viruses; | Monocytes/macrophages |
| | RNA-immunoglobulin | Plasmacytoid dendritic cells |
| | complexes | B lymphocytes |
| TLR8 | single stranded RNA viruses; | Monocytes/macrophages |
| | RNA-immunoglobulin | Dendritic cells |
| | complexes | Mast cells |
| TLR9 | DNA containing unmethylated | Monocytes/macrophages |
| | “CpG” motifs; DNA- | Plasmacytoid dendritic cells |
| | immunoglobulin complexes | B lymphocytes |

The signal transduction pathway mediated by the interaction between a ligand and a TLR is shared among most members of the TLR family and involves a toll/IL-1 receptor (TIR domain), the myeloid differentiation marker 88 (MyD88), IL-1R-associated kinase (IRAK), interferon regulating factor (IRF), TNF-receptor-associated factor (TRAF), TGFβ-activated kinasel, IκB kinases, IκB, and NF-κB (see for example: Akira, S. (2003) J. Biol. Chem. 278:38105 and Geller at al. (2008) Curr. Drug Dev. Tech. 5:29-38). More specifically, for TLRs 1, 2, 4, 5, 6, 7, 8, 9 and 11, this signaling cascade begins with a PAMP ligand interacting with and activating the membrane-bound TLR, which exists as a homo-dimer in the endosomal membrane or the cell surface. Following activation, the receptor undergoes a conformational change to allow recruitment of the TIR domain containing protein MyD88, which is an adapter protein that is common to all TLR signaling pathways except TLR3. MyD88 recruits IRAK4, which phosphorylates and activates IRAK1. The activated IRAK1 binds with TRAF6, which catalyzes the addition of polyubiquitin onto TRAF6. The addition of ubiquitin activates the TAK/TAB complex, which in turn phosphorylates IRFs, resulting in NF-kB release and transport to the nucleus. NF-kB in the nucleus induces the expression of pro-inflammatory genes (see for example, Trinchieri and Sher (2007) Nat. Rev. Immunol. 7:179-190).

Certain unmethylated CpG motifs present in bacterial and synthetic DNA have been shown to activate the immune system and induce antitumor activity. (Tokunaga T et al., J. Natl. Cancer Inst. (1984) 72:955-962; Shimada S, et al., Jpn. H cancer Res, 1986, 77, 808-816; Yamamoto S, et al., Jpn. J. Cancer Res., 1986, 79, 866-73). During the development of antisense technology, it was discovered that oligonucleotides containing unmethylated CpG dinucleotides stimulate immune responses (Zhao Q, et al. (1996) Biochem. Pharmacol. 26:173-182). Subsequent studies demonstrated that TLR9 recognizes unmethylated CpG motifs present in bacterial and synthetic DNA (Hemmi, H. et al. (2000) Nature 408:740-745). Detailed structure-activity relationship studies have elucidated that in addition to unmethylated CpG motifs, chemical modifications in the sequence flanking the CpG motif alter the immune stimulatory activity of the oligonucleotide. (see for example: Zhao et al., Biochem. Pharmacol. (1996) 51:173-182; Zhao et al. (1996) Biochem Pharmacol. 52:1537-1544; Zhao et al. (1997) Antisense Nucleic Acid Drug Dev. 7:495-502; Zhao et al (1999) Bioorg. Med. Chem. Lett. 9:3453-3458; Zhao et al. (2000) Bioorg. Med. Chem. Lett. 10:1051-1054; Yu, D. et al. (2000) Bioorg. Med. Chem. Lett. 10:2585-2588; Yu, D. et al. (2001) Bioorg. Med. Chem. Lett. 11:2263-2267; and Kandimalla, E. et al. (2001) Bioorg. Med. Chem. 9:807-813). Certain CpG-containing oligonucleotides have been shown to produce anti-tumor activity (e.g. tumor growth and angiogenesis) resulting in an effective anti-cancer response (e.g. anti-leukemia) (Smith, J. B. and Wickstrom, E. (1998) J. Natl. Cancer Inst. 90:1146-1154). In addition, TLR9 agonists have been shown to work synergistically with other known anti-tumor compounds (e.g. cetuximab, irinotecan) (Vincenzo, D., et al. (2006) Clin. Cancer Res. 12(2):577-583).

The selective localization of TLRs and the signaling generated therefrom, provides some insight into their role in the immune response. The immune response involves both an innate and an adaptive response based upon the subset of cells involved in the response. For example, the T helper (Th) cells involved in classical cell-mediated functions such as delayed-type hypersensitivity and activation of cytotoxic T lymphocytes (CTLs) are Th1 cells. This response is the body's innate response to antigen (e.g. viral infections, intracellular pathogens, and tumor cells), and results in a secretion of IFN-gamma and a concomitant activation of CTLs.

As a result of their involvement in regulating an inflammatory response, TLRs have been shown to play a role in the pathogenesis of many diseases, including autoimmunity, infectious disease and inflammation (Papadimitraki et al. (2007) J. Autoimmun. 29: 310-318; Sun et al. (2007) Inflam. Allergy Drug Targets 6:223-235; Diebold (2008) Adv. Drug Deliv. Rev. 60:813-823; Cook, D. N. et al. (2004) Nature Immunol. 5:975-979; Tse and Horner (2008) Semin. Immunopathol. 30:53-62; Tobias & Curtiss (2008) Semin. Immunopathol. 30:23-27; Ropert et al. (2008) Semin. Immunopathol. 30:41-51; Lee et al. (2008) Semin. Immunopathol. 30:3-9; Gao et al. (2008) Semin. Immunopathol. 30:29-40; Vijay-Kumar et al. (2008) Semin. Immunopathol. 30:11-21). While activation of TLRs is involved in mounting an immune response, an uncontrolled or undesired stimulation of the immune system through TLRs may exacerbate certain diseases in immune compromised subjects or may cause unwanted immune stimulation. Thus, down-regulating TLR expression and/or activity may provide a useful means for disease intervention.

To date, investigative strategies aimed selectively at inhibiting TLR activity have involved small molecules (WO/2005/007672), antibodies (see for example: Duffy, K. et al. (2007) Cell Immunol. 248:103-114), catalytic RNAi technologies (e.g. small inhibitory RNAs), certain antisense molecules (Caricilli et al. (2008) J. Endocrinology 199:399), and competitive inhibition with modified or methylated oligonucleotides (see for example: Kandimalla et al. US2008/0089883; Barrat and Coffman (2008) Immunol. Rev. 223:271-283). For example, chloroquine and hydroxylchloroquine have been shown to block endosomal-TLR signaling by down-regulating the maturation of endosomes (Krieg, A. M. (2002) Annu. Rev. Immunol. 20:709). Also, Huang et al. have shown the use of TLR4 siRNA to reverse the tumor-mediated suppression of T cell proliferation and natural killer cell activity (Huang et al. (2005) Cancer Res. 65:5009-5014), and the use of TLR9 siRNA to prevent bacterial-induced inflammation of the eye (Huang et al. (2005) Invest. Opthal. Vis. Sci. 46:4209-4216).

Additionally, several groups have used synthetic oligodeoxynucleotides having two triplet sequences, a proximal “CCT” triplet and a distal “GGG” triplet, a poly “G” (e.g. “GGGG” or “GGG”) or “GC” sequences that interact with certain intracellular proteins, resulting in the inhibition of TLR signaling and the concomitant production and release of pro-inflammatory cytokines (see for example: Lenert, P. et al. (2003) DNA Cell Biol. 22(10):621-631; Patole, P. et al. (2005) J. Am. Soc. Nephrol. 16:3273-3280), Gursel, I., et al. (J. Immunol., 171: 1393-1400 (2003), Shirota, H., et al., J. Immunol., 173: 5002-5007 (2004), Chen, Y., et al., Gene Ther. 8: 1024-1032 (2001); Stunz, L. L., Eur. J. Immunol. (2000) 32: 1212-1222; Kandimalla et al. WO2007/7047396). However, oligonucleotides containing guanosine strings have been shown to form tetraplex structures, act as aptamers and inhibit thrombin activity (Bock L C et al., Nature, 355:564-6, 1992; Padmanabhan, K et al., J Biol. Chem., 268(24):17651-4, 1993). Thus, the utility of these inhibitory oligodeoxynucleotide molecules may not be achievable in patients.

As an alternative to interacting with the receptor protein and directly inhibiting receptor activation, some studies have suggested the utility of “knock down” or silencing technologies, for example siRNA, miRNA, ddRNA and eiRNA technologies, for inhibiting the activity of a receptor. These technologies rely upon administration or expression of double stranded RNA (dsRNA). However, RNAi molecules act through a catalytic process, these molecules are recognized as being distinct from other technologies that target RNA molecules and inhibit their translation (see for example: Opalinska and Gewirtz (2002) Nature Reviews 1:503-514). Moreover, siRNA molecules have been recognized to induce non-specific immune stimulation through interaction with TLRs (Kleinman et al., (2008) Nature 452:591-597; De Veer et. al. (2005) Immun. Cell Bio. 83:224-228; Kariko et al. (2004) J. Immunol. 172:6545-6549).

A promising approach to suppressing the activity of TLR9 is the use of oligonucleotide-based antagonists (see Kandimalla et al., WO2007/7047396).

Yet another potential approach to “knock down” expression of TLRs is antisense technology. The history of antisense technology has revealed that while discovery of antisense oligonucleotides that inhibit gene expression is relatively straight forward, the optimization of antisense oligonucleotides that have true potential as clinical candidates is not. Accordingly, if an antisense approach to down-regulating TLR9 is to be successful, there is a need for optimized antisense oligonucleotides that most efficiently achieve this result. Such optimized antisense oligonucleotides could be used alone, or in conjunction with the antagonists of Kandimalla et al., or other therapeutic approaches.

### BRIEF SUMMARY OF THE INVENTION

The present invention is directed to optimized synthetic antisense oligonucleotides that are targeted to a nucleic acid encoding TLR9 and that efficiently inhibit the expression of TLR9 through inhibition of mRNA translation and/or through an RNase H mediated mechanism.

In a first aspect, the invention provides for optimized antisense oligonucleotides including those having SEQ ID NOs: 3, 4, 7, 18, 41, 42, 49, 55, 65, 81, 83, 87, 116, 125, 159, 167 or 189.

In a second aspect, the invention provides a composition comprising at least one optimized antisense oligonucleotide according to the invention and a physiologically acceptable carrier, diluent or excipient.

In a third aspect, the invention provides a method of inhibiting TLR9 expression. In this method, an oligonucleotide or multiple oligonucleotides of the invention are specifically contacted or hybridized with TLR9 mRNA either in vitro or in a cell.

In a fourth aspect, the invention provides methods for inhibiting the expression of TLR9 in a mammal, particularly a human, such methods comprising administering to the mammal a compound or composition according to the invention.

In a fifth aspect, the invention provides a method for inhibiting a TLR9-mediated immune response in a mammal, the method comprising administering to the mammal a TLR9 antisense oligonucleotide according to the invention in a pharmaceutically effective amount.

In a sixth aspect, the invention provides a method for therapeutically treating a mammal having a disease mediated by TLR9, such method comprising administering to the mammal, particularly a human, a TLR9 antisense oligonucleotide of the invention, or a composition thereof, in a pharmaceutically effective amount.

In a seventh aspect, the invention provides methods for preventing a disease or disorder in a mammal, particularly a human, at risk of contracting or developing a disease or disorder mediated by TLR9. The method according to this aspect of the invention comprises administering to the mammal an antisense oligonucleotide according to the invention, or a composition thereof, in a prophylactically effective amount.

In an eighth aspect, the invention provides methods for down-regulating TLR9 expression and thus preventing the “off-target” activity of certain other antisense molecules, or other compounds or drugs that have a side effect of activating TLR9. For example, the TLR9 antisense oligonucleotide according to the invention can be administered in combination with one or more antisense oligonucleotides or other nucleic acid containing compounds, which are not the same target as the antisense molecule of the invention, and which comprise an immunostimulatory motif that would activate a TLR9-mediated immune response but for the presence of the TLR9 antisense oligonucleotide according to the invention.

The subject oligonucleotides and methods of the invention are also useful for examining the function of the TLR9 gene in a cell or in a control mammal or in a mammal afflicted with a disease associated with TLR9 or immune stimulation through TLR9. The cell or mammal is administered the oligonucleotide, and the expression of TLR9 mRNA or protein is examined.

### BRIEF DESCRIPTION OF THE DRAWINGS

FIG. 1 is a synthetic scheme for the linear synthesis of immune modulatory compounds of the invention. DMTr=4,4′-dimethoxytrityl; CE=cyanoethyl.

FIG. 2 is a graphic representation of the activity of exemplary mouse TLR9 antisense oligonucleotide according to the invention in HEK293 cells expressing mouse TLR9. The data demonstrate the ability of exemplar oligonucleotides according to the invention to inhibit TLR9 expression and activation in HEK293 cells that were cultured and treated according to Example 2.

FIG. 3 is a graphical representation of the activity of exemplar human TLR9 antisense oligonucleotides according to the invention in HEK293XL cells expressing human TLR9. The data demonstrate the ability of exemplar oligonucleotides according to the invention to inhibit TLR9 expression and activation in HEK293 cells that were cultured and treated according to Example 2.

FIG. 4 is a graphical representation of the activity of exemplar TLR9 antisense oligonucleotides according to the invention to inhibit TLR9 expression and downstream cytokine and chemokine release and activity in human PBMCs. The data demonstrate the ability of exemplar oligonucleotides according to the invention to inhibit TLR9 expression and the downstream cytokine and chemokine release and activity in PBMC that were cultured and treated according to Example 3.

FIG. 5 is a graphical representation of the activity of exemplar TLR9 antisense oligonucleotides according to the invention to inhibit TLR9 expression in mouse spleen following in vivo administration or in human PBMCs following in vitro administration. The data demonstrate that administration of an exemplar TLR9 antisense oligonucleotide according to the invention can cause down-regulation of TLR9 expression in vivo and in vitro.

FIG. 6 is a graphical representation of the activity of exemplar TLR9 antisense oligonucleotides according to the invention to inhibit TLR9-induced IL-12 following in vivo administration. The data demonstrate that administration of an exemplar TLR9 antisense oligonucleotide according to the invention can cause down-regulation of TLR9 expression in vivo and prevent the induction of IL-12 by a TLR9 agonist. More generally, the data demonstrate the ability of a TLR9 antisense oligonucleotide according to the invention to inhibit the induction of pro-inflammatory cytokines by a TLR9 agonist.

FIGS. 7a and 7b are graphical representations of the activity of exemplar TLR9 antisense oligonucleotides according to the invention to inhibit psoriasis in vivo. The data demonstrate that administration of an exemplar TLR9 antisense oligonucleotide according to the invention can inhibit epidermal hyperplasia and leukocyte infiltration in IL-23 induced psoriatic lesions. More generally, the data demonstrate the ability of TLR9 antisense oligonucleotides according to the invention to inhibit TLR9-mediated diseases in vivo, including without limitation, psoriasis.

FIG. 8 depicts human TLR9 mRNA (SEQ ID NO: 206)(GenBank Accession No. AAF78037).

### DETAILED DESCRIPTION OF THE PREFERRED EMBODIMENTS

The invention relates to optimized TLR9 antisense oligonucleotides, compositions comprising such oligonucleotides and methods of their use for inhibiting or suppressing a TLR9-mediated immune response. The antisense oligonucleotides according to the invention are stable, specific and do not activate an innate immune response, thereby overcoming the problems of certain previously attempted approaches. Pharmaceutical and other compositions comprising the compounds according to the invention are also provided. Further provided are methods of down-regulating the expression of TLR9 in cells or tissues comprising contacting said cells or tissues with one or more of the antisense compounds or compositions of the invention alone or in combination with other prophylactic or therapeutic compositions.

Specifically, the invention provides antisense oligonucleotides designed to be complementary to a genomic region or an RNA molecule transcribed therefrom. These TLR9 antisense oligonucleotides have unique sequences that target specific, particularly available mRNA sequences, resulting in maximally effective inhibition or suppression of TLR9-mediated signaling in response to endogenous and/or exogenous TLR9 ligands or TLR9 agonists.

The TLR9 antisense oligonucleotides according to the invention inhibit immune responses induced by natural or artificial TLR9 agonists in various cell types and in various in vitro and in vivo experimental models. As such, the antisense compositions according to the invention are useful as tools to study the immune system, as well as to compare the immune systems of various animal species, such as humans and mice.

Further provided are methods of treating an animal, particularly a human, having, suspected of having, or being prone to develop a disease or condition associated with TLR9 activation by administering a therapeutically or prophylactically effective amount of one or more of the antisense compounds or compositions of the invention. These can be used for immunotherapy applications such as, but not limited to, treatment of cancer, autoimmune disorders, asthma, respiratory allergies, food allergies, skin allergies, systemic lupus erythematosus (SLE), arthritis, pleurisy, chronic infections, inflammatory diseases, inflammatory bowel syndrome, sepsis, malaria, and bacteria, parasitic, and viral infections in adult and pediatric human and veterinary applications. In addition, The TLR9 antisense oligonucleotides according to the invention are also useful in the prevention and/or treatment of various diseases, either alone, in combination with or co-administered with other drugs or prophylactic or therapeutic compositions, for example, DNA vaccines, antigens, antibodies, and allergens; and in combination with chemotherapeutic agents (both traditional chemotherapy and modern targeted therapies) and/or TLR9 antagonists for prevention and treatment of diseases. TLR9 antisense oligonucleotides of the invention are useful in combination with compounds or drugs that have unwanted TLR9-mediated immune stimulatory properties.

The patents and publications cited herein reflect the level of knowledge in the art and are hereby incorporated by reference in their entirety. Any conflict between the teachings of these patents and publications and this specification shall be resolved in favor of the latter.

The foregoing and other objects of the present invention, the various features thereof, as well as the invention itself may be more fully understood from the following description, when read together with the accompanying drawings in which:

The term “2′-O-substituted” means substitution of the 2′ position of the pentose moiety with an —O— lower alkyl group containing 1-6 saturated or unsaturated carbon atoms (for example, but not limited to, 2′-O-methyl), or with an —O-aryl or allyl group having 2-6 carbon atoms, wherein such alkyl, aryl or allyl group may be unsubstituted or may be substituted, (for example, with 2′-O-ethoxy-methyl, halo, hydroxy, trifluoromethyl, cyano, nitro, acyl, acyloxy, alkoxy, carboxyl, carbalkoxyl, or amino groups); or with a hydroxy, an amino or a halo group, but not with a 2′-H group. In some embodiments the oligonucleotides of the invention include four or five ribonucleotides 2′-O-alkylated at their 5′ terminus (i.e., 5′ 2-O-alkylated ribonucleotides), and/or four or five ribonucleotides 2′-O-alkylated at their 3′ terminus (i.e., 3′ 2-O-alkylated ribonucleotides). In exemplar embodiments, the nucleotides of the synthetic oligonucleotides are linked by at least one phosphorothioate internucleotide linkage. The phosphorothioate linkages may be mixed Rp and Sp enantiomers, or they may be stereoregular or substantially stereoregular in either Rp or Sp form (see Iyer et al. (1995) Tetrahedron Asymmetry 6:1051-1054).

The term “3′”, when used directionally, generally refers to a region or position in a polynucleotide or oligonucleotide 3′ (toward the 3′ end of the nucleotide) from another region or position in the same polynucleotide or oligonucleotide.

The term “5′”, when used directionally, generally refers to a region or position in a polynucleotide or oligonucleotide 5′ (toward the 5′ end of the nucleotide) from another region or position in the same polynucleotide or oligonucleotide.

The term “about” generally means that the exact number is not critical. Thus, oligonucleotides having one or two fewer nucleoside residues, or from one to several additional nucleoside residues are contemplated as equivalents of each of the embodiments described above.

The term “agonist” generally refers to a substance that binds to a receptor of a cell and induces a response. An agonist often mimics the action of a naturally occurring substance such as a ligand.

The term “antagonist” generally refers to a substance that attenuates the effects of an agonist.

The term “airway inflammation” generally includes, without limitation, inflammation in the respiratory tract caused by allergens, including asthma.

The term “allergen” generally refers to an antigen or antigenic portion of a molecule, usually a protein, which elicits an allergic response upon exposure to a subject. Typically the subject is allergic to the allergen as indicated, for instance, by the wheal and flare test or any method known in the art. A molecule is said to be an allergen even if only a small subset of subjects exhibit an allergic (e.g., IgE) immune response upon exposure to the molecule.

The term “allergy” generally includes, without limitation, food allergies, respiratory allergies and skin allergies.

The term “antigen” generally refers to a substance that is recognized and selectively bound by an antibody or by a T cell antigen receptor. Antigens may include but are not limited to peptides, proteins, nucleosides, nucleotides and combinations thereof. Antigens may be natural or synthetic and generally induce an immune response that is specific for that antigen.

The term “autoimmune disorder” generally refers to disorders in which “self” antigen undergo attack by the immune system. Such term includes, without limitation, lupus erythematosus, multiple sclerosis, type I diabetes mellitus, irritable bowel syndrome, Chron's disease, rheumatoid arthritis, septic shock, alopecia universalis, acute disseminated encephalomyelitis, Addison's disease, ankylosing spondylitis, antiphospholipid antibody syndrome, autoimmune hemolytic anemia, autoimmune hepatitis, Bullous pemphigoid, chagas disease, chronic obstructive pulmonary disease, coeliac disease, dermatomyositis, endometriosis, Goodpasture's syndrome, Graves' disease, Guillain-Barrésyndrome, Hashimoto's disease, hidradenitis suppurativa, idiopathic thrombocytopenic purpura, interstitial cystitis, morphea, myasthenia gravis, narcolepsy, neuromyotonia, pemphigus, pernicious anaemia, polymyositis, primary biliary cirrhosis, schizophrenia, Sjögren's syndrome, temporal arteritis (“giant cell arteritis”), vasculitis, vitiligo, vulvodynia and Wegener's granulomatosis autoimmune asthma, septic shock and psoriasis.

The term “cancer” generally refers to, without limitation, any malignant growth or tumor caused by abnormal or uncontrolled cell proliferation and/or division. Cancers may occur in humans and/or animals and may arise in any and all tissues. Treating a patient having cancer may include administration of a compound, pharmaceutical formulation or vaccine according to the invention such that the abnormal or uncontrolled cell proliferation and/or division, or metastasis is affected.

The term “carrier” generally encompasses any excipient, diluent, filler, salt, buffer, stabilizer, solubilizer, oil, lipid, lipid containing vesicle, microspheres, liposomal encapsulation, or other material well known in the art for use in pharmaceutical formulations. It will be understood that the characteristics of the carrier, excipient, or diluent will depend on the route of administration for a particular application. The preparation of pharmaceutically acceptable formulations containing these materials is described in, for example, Remington 's Pharmaceutical Sciences, 18th Edition, ed. A. Gennaro, Mack Publishing Co., Easton, Pa., 1990.

The term “co-administration” or “co-administered” generally refers to the administration of at least two different substances sufficiently close in time to modulate an immune response. Co-administration refers to simultaneous administration, as well as temporally spaced order of up to several days apart, of at least two different substances in any order, either in a single dose or separate doses.

The term “in combination with” generally means administering a compound according to the invention and another agent useful for treating the disease or condition that does not abolish TLR9 antisense activity of the compound in the course of treating a patient. Such administration may be done in any order, including simultaneous administration, as well as temporally spaced order from a few seconds up to several days apart. Such combination treatment may also include more than a single administration of the compound according to the invention and/or independently the other agent. The administration of the compound according to the invention and the other agent may be by the same or different routes.

The term “individual” or “subject” or “vertebrate” generally refers to a mammal, such as a human.

The term “linear synthesis” generally refers to a synthesis that starts at one end of an oligonucleotide and progresses linearly to the other end. Linear synthesis permits incorporation of either identical or non-identical (in terms of length, base composition and/or chemical modifications incorporated) monomeric units into an oligonucleotide.

The term “mammal” is expressly intended to include warm blooded, vertebrate animals, including, without limitation, humans, non-human primates, rats, mice, cats, dogs, horses, cattle, cows, pigs, sheep and rabbits.

The term “nucleoside” generally refers to compounds consisting of a sugar, usually ribose or deoxyribose, and a purine or pyrimidine base.

The term “nucleotide” generally refers to a nucleoside comprising a phosphorous-containing group attached to the sugar.

The term “modified nucleoside” generally is a nucleoside that includes a modified heterocyclic base, a modified sugar moiety, or any combination thereof. In some embodiments, the modified nucleoside is a non-natural pyrimidine or purine nucleoside, as herein described. For purposes of the invention, a modified nucleoside, a pyrimidine or purine analog or non-naturally occurring pyrimidine or purine can be used interchangeably and refers to a nucleoside that includes a non-naturally occurring base and/or non-naturally occurring sugar moiety. For purposes of the invention, a base is considered to be non-natural if it is not guanine, cytosine, adenine, thymine or uracil and a sugar is considered to be non-natural if it is not β-ribo-furanoside or 2′-deoxyribo-furanoside.

The term “modified oligonucleotide” as used herein describes an oligonucleotide in which at least two of its nucleotides are covalently linked via a synthetic linkage, i.e., a linkage other than a phosphodiester linkage between the 5′ end of one nucleotide and the 3′ end of another nucleotide in which the 5′ nucleotide phosphate has been replaced with any number of chemical groups. The term “modified oligonucleotide” also encompasses oligonucleotides having at least one nucleotide with a modified base and/or sugar, such as a 2′-O-substituted, a 5′-O-substituted and/or a 3′-O-substituted ribonucleotide.

The term “nucleic acid” encompasses a genomic region or an RNA molecule transcribed therefrom. In some embodiments, the nucleic acid is mRNA.

The term “nucleotidic linkage” generally refers to a chemical linkage to join two nucleosides through their sugars (e.g. 3′-3′,2′-3′,2′-5′,3′-5′) consisting of a phosphorous atom and a charged, or neutral group (e.g., phosphodiester, phosphorothioate, phosphorodithioate or methylphosphonate) between adjacent nucleosides.

The term “oligonucleotide” refers to a polynucleoside formed from a plurality of linked nucleoside units. The nucleoside units may be part of viruses, bacteria, cell debris or oligonucleotide-based compositions (for example, siRNA and microRNA). Such oligonucleotides can also be obtained from existing nucleic acid sources, including genomic or cDNA, but are preferably produced by synthetic methods. In certain embodiments each nucleoside unit includes a heterocyclic base and a pentofuranosyl, trehalose, arabinose, 2′-deoxy-2′-substituted nucleoside, 2′-deoxy-2′-substituted arabinose, 2′-O-substituted arabinose or hexose sugar group. The nucleoside residues can be coupled to each other by any of the numerous known internucleoside linkages. Such internucleoside linkages include, without limitation, phosphodiester, phosphorothioate, phosphorodithioate, methylphosphonate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone internucleoside linkages. The term “oligonucleotide-based compound” also encompasses polynucleosides having one or more stereospecific internucleoside linkage (e.g., (R_{P})- or (S_{P})-phosphorothioate, alkylphosphonate, or phosphotriester linkages). As used herein, the terms “oligonucleotide” and “dinucleotide” are expressly intended to include polynucleosides and dinucleosides having any such internucleoside linkage, whether or not the linkage comprises a phosphate group. In certain exemplar embodiments, these internucleoside linkages may be phosphodiester, phosphorothioate or phosphorodithioate linkages, or combinations thereof.

The term “complementary to a genomic region or an RNA molecule transcribed therefrom” is intended to mean an oligonucleotide that binds to the nucleic acid sequence under physiological conditions, for example, by Watson-Crick base pairing (interaction between oligonucleotide and single-stranded nucleic acid) or by Hoogsteen base pairing (interaction between oligonucleotide and double-stranded nucleic acid) or by any other means, including in the case of an oligonucleotide, binding to RNA and causing pseudoknot formation. Binding by Watson-Crick or Hoogsteen base pairing under physiological conditions is measured as a practical matter by observing interference with the function of the nucleic acid sequence.

The term “peptide” generally refers to polypeptides that are of sufficient length and composition to affect a biological response, for example, antibody production or cytokine activity whether or not the peptide is a hapten. The term “peptide” may include modified amino acids (whether or not naturally or non-naturally occurring), where such modifications include, but are not limited to, phosphorylation, glycosylation, pegylation, lipidization and methylation.

The term “pharmaceutically acceptable” means a non-toxic material that does not interfere with the effectiveness of a compound according to the invention or the biological activity of a compound according to the invention.

The term “physiologically acceptable” refers to a non-toxic material that is compatible with a biological system such as a cell, cell culture, tissue, or organism. Preferably, the biological system is a living organism, such as a vertebrate, including a mammal, particularly a human.

The term “prophylactically effective amount” generally refers to an amount sufficient to prevent or reduce the development of an undesired biological effect.

The term “therapeutically effective amount” or “pharmaceutically effective amount” generally refers to an amount sufficient to affect a desired biological effect, such as a beneficial result, including, without limitation, prevention, diminution, amelioration or elimination of signs or symptoms of a disease or disorder. Thus, the total amount of each active component of the pharmaceutical composition or method is sufficient to show a meaningful patient benefit, for example, but not limited to, healing of chronic conditions characterized by immune stimulation. Thus, a “pharmaceutically effective amount” will depend upon the context in which it is being administered. A pharmaceutically effective amount may be administered in one or more prophylactic or therapeutic administrations. When applied to an individual active ingredient, administered alone, the term refers to that ingredient alone. When applied to a combination, the term refers to combined amounts of the active ingredients that result in the therapeutic effect, whether administered in combination, serially or simultaneously.

The term “treatment” generally refers to an approach intended to obtain a beneficial or desired result, which may include alleviation of symptoms, or delaying or ameliorating a disease progression.

In a first aspect, the invention provides antisense oligonucleotides that are complementary to a nucleic acid that is specific for human TLR9 (SEQ ID NO: 206). The antisense oligonucleotides according to the invention are optimized with respect to the targeted region of the TLR9 mRNA coding sequence or 5′ untranslated region or the 3′ untranslated region, in their chemical modification and/or both. In some embodiments of this aspect, the compounds are complementary to a region within nucleobases 635 through 3730 of the coding region, or 1-634 of the 5′ untranslated region of TLR9 mRNA, or 3731 through 3868 of the 3′ untranslated region. (SEQ ID NO: 206).

Antisense oligonucleotides according to the invention are useful in treating and/or preventing diseases wherein inhibiting a TLR9-mediated immune response would be beneficial. TLR9-targeted antisense oligonucleotides according to the invention that are useful include, but are not limited to, antisense oligonucleotides comprising naturally occurring nucleotides, modified nucleotides, modified oligonucleotides and/or backbone modified oligonucleotides. However, antisense oligonucleotides that inhibit the translation of mRNA encoded proteins may produce undesired biological effects, including but not limited to insufficiently active antisense oligonucleotides, inadequate bioavailability, suboptimal pharmacokinetics or pharmacodynamics, and immune stimulation. Thus, the optimal design of an antisense oligonucleotide according to the invention requires many, non-obvious considerations beyond simple design of a complementary sequence. Thus, preparation of TLR9-targeted antisense oligonucleotides according to the invention is intended to incorporate changes necessary to limit secondary structure interference with antisense activity, enhance the oligonucleotide's target specificity, minimize interaction with binding or competing factors (for example, proteins), optimize cellular uptake, stability, bioavailability, pharmacokinetics and pharmacodynamics, and/or inhibit, prevent or suppress immune cell activation. Such inhibition, prevention or suppression of immune cell activation may be accomplished in a number of ways without compromising the antisense oligonucleotide's ability to hybridize to nucleotide sequences contained within the mRNA for TLR9, including, without limitation, incorporation of one or more modified nucleotides or nucleotide linkages, wherein such modified nucleotides are a 2′-O-methyl, a 3′-O-methyl, a 5-methyl, a 2′-O-methoxyethyl-C, a 2′-O-methoxyethyl-5-methyl-C and/or a 2′-O-methyl-5-methyl-C on the “C” of a “CpG” dinucleotide, a 2′-O-substituted-G, a 2′-O-methyl-G and/or a 2′-O-methoxyethoxy-G on the “G” of the CpG, and such modified nucleotide linkages are a non-phosphate or non-phosphorothioate internucleoside linkage between the C and G of a “CpG” dinucleotide, a methylphosphonate linkage and/or a 2′-5′ internucleotide linkage between the C and G of a “CpG” dinucleotide.

It has been determined that the TLR9 coding region is comprised of 3.1 kB, and the transcript corresponding to the 1032 amino acid protein have also been identified in humans (Chuang and Ulevitch, Eur. Cytokine Network (2000) 3:372-378). The sequence of the gene encoding TLR9 has been reported in mice (Hemmi et al. (2000) 408:740-745) and for humans (Chuang and Ulevitch, Eur. Cytokine Network (2000) 3:372-378). The oligonucleotides of the invention are directed to optimally available portions of the TLR9 nucleic acid sequence that most effectively act as a target for inhibiting TLR9 expression. These targeted regions of the TLR9 gene include portions of the known exons or 5′ untranslated region. In addition, intron-exon boundaries, 3′ untranslated regions and introns are potentially useful targets for antisense inhibition of TLR9 expression. The nucleotide sequences of some representative, non-limiting oligonucleotides specific for human TLR9 have SEQ ID NOS: 1-205. The nucleotide sequences of optimized oligonucleotides according to the invention include those having SEQ ID NOS: 3, 4, 7, 18, 41, 42, 49, 55, 65, 81, 83, 87, 116, 125, 159, 167 or 189.

The oligonucleotides of the invention are composed of ribonucleotides, deoxyribonucleotides or a combination of both, with the 5′ end of one nucleotide and the 3′ (or in limited cases 2′) end of another nucleotide being covalently linked. These oligonucleotides are at least 14 nucleotides in length, but are preferably 15 to 60 nucleotides long, preferably 20 to 50 nucleotides in length. In some embodiments, these oligonucleotides contain from about 14 to 28 nucleotides or from about 16 to 25 nucleotides or from about 18 to 22 nucleotides or 20 nucleotides. These oligonucleotides can be prepared by the art recognized methods such as phosphoramidate or H-phosphonate chemistry which can be carried out manually or by an automated synthesizer. The synthetic TLR9 antisense oligonucleotides of the invention may also be modified in a number of ways without compromising their ability to hybridize to TLR9 mRNA. Such modifications may include at least one internucleotide linkage of the oligonucleotide being an alkylphosphonate, phosphorothioate, phosphorodithioate, methylphosphonate, phosphate ester, alkylphosphonothioate, phosphoramidate, carbamate, carbonate, phosphate triester, acetamidate or carboxymethyl ester or a combination of these and other internucleotide linkages between the 5′ end of one nucleotide and the 3′ end of another nucleotide in which the 5′ nucleotide phosphodiester linkage has been replaced with any number of chemical groups.

For example, U.S. Pat. No. 5,149,797 describes traditional chimeric oligonucleotides having a phosphorothioate core region interposed between methylphosphonate or phosphoramidate flanking regions. U.S. Pat. No. 5,652,356 discloses “inverted” chimeric oligonucleotides comprising one or more nonionic oligonucleotide region (e.g. alkylphosphonate and/or phosphoramidate and/or phosphotriester internucleoside linkage) flanked by one or more region of oligonucleotide phosphorothioate. Various oligonucleotides with modified internucleotide linkages can be prepared according to standard methods, Phosphorothioate linkages may be mixed Rp and Sp enantiomers, or they may be made stereoregular or substantially stereoregular in either Rp or Sp form according to standard procedures.

Oligonucleotides which are self-stabilized are also considered to be modified oligonucleotides useful in the methods of the invention (Tang et al. (1993) Nucleic Acids Res. 20:2729-2735). These oligonucleotides comprise two regions: a target hybridizing region; and a self-complementary region having an oligonucleotide sequence complementary to a nucleic acid sequence that is within the self-stabilized oligonucleotide.

Other modifications include those which are internal or at the end(s) of the oligonucleotide molecule and include additions to the molecule of the internucleoside phosphate linkages, such as cholesterol, cholesteryl, or diamine compounds with varying numbers of carbon residues between the amino groups and terminal ribose, deoxyribose and phosphate modifications which cleave, or crosslink to the opposite chains or to associated enzymes or other proteins which bind to the genome. Examples of such modified oligonucleotides include oligonucleotides with a modified base and/or sugar such as arabinose instead of ribose, or a 3′,5′-substituted oligonucleotide having a sugar which, at both its 3′ and 5′ positions, is attached to a chemical group other than a hydroxyl group (at its 3′ position) and other than a phosphate group (at its 5′ position).

Other examples of modifications to sugars include modifications to the 2′ position of the ribose moiety which include but are not limited to 2′-O-substituted with an —O-alkyl group containing 1-6 saturated or unsaturated carbon atoms, or with an —O-aryl, or —O-allyl group having 2-6 carbon atoms wherein such —O-alkyl, —O-aryl or —O-allyl group may be unsubstituted or may be substituted, for example with halo, hydroxy, trifluoromethyl cyano, nitro acyl acyloxy, alkoxy, carboxy, carbalkoxyl or amino groups. None of these substitutions are intended to exclude the native 2′-hydroxyl group in the case of ribose or 2′1-H— in the case of deoxyribose.

U.S. Pat. No. 5,652,355 discloses traditional hybrid oligonucleotides having regions of 2′-O-substituted ribonucleotides flanking a DNA core region. U.S. Pat. No. 5,652,356 discloses an “inverted” hybrid oligonucleotide which includes an oligonucleotide comprising a 2′-O-substituted (or 2′ OH, unsubstituted) RNA region which is in between two oligodeoxyribonucleotide regions, a structure that “inverted relative to the “traditional” hybrid oligonucleotides. Non-limiting examples of particularly useful oligonucleotides of the invention have 2′-O-alkylated ribonucleotides at their 3′, 5′, or 3′ and 5′ termini, with at least four or five contiguous nucleotides being so modified. Non-limiting examples of 2′-O-alkylated groups include 2′-O-methyl, 2′-O-ethyl, 2′-O-propyl, 2′-O-butyls and 2′-O-ethoxy-methyl.

Other modified oligonucleotides are capped with a nuclease resistance-conferring bulky substituent at their 3′ and/or 5′ end(s), or have a substitution in one non-bridging oxygen per nucleotide. Such modifications can be at some or all of the internucleoside linkages, as well as at either or both ends of the oligonucleotide and/or in the interior of the molecule.

The oligonucleotides of the invention can be administered in combination with one or more antisense oligonucleotides or other nucleic acid containing compounds, which are not the same target as the antisense molecule of the invention, and which comprise an immunostimulatory motif that would activate a TLR9-mediated immune response but for the presence of the TLR9 antisense oligonucleotide according to the invention. In addition, the oligonucleotides of the invention can be administered in combination with one or more vaccines, antigens, antibodies, cytotoxic agents, allergens, antibiotics, TLR antagonists, siRNA, miRNA, antisense oligonucleotides, aptamers, peptides, proteins, gene therapy vectors, DNA vaccines, adjuvants, kinase inhibitors or co-stimulatory molecules or combinations thereof.

A non-limiting list of TLR9 antisense oligonucleotides are shown in SEQ ID NO. 1 through SEQ ID NO 205 and Table 2 below. Optimized antisense oligonucleotides according to the invention include those having SEQ ID NOS: 3, 4, 7, 18, 41, 42, 49, 55, 65, 81, 83, 87, 116, 125, 159, 167 or 189. In Table 2, the oligonucleotide-based TLR9 antisense compounds have all phosphorothioate (PS) linkages, except where indicated. Those skilled in the art will recognize, however, that phosphodiester (PO) linkages, or a mixture of PS and PO linkages can be used.

TABLE 2

| SEQ | Position | Antisense Sequence. |
|---|---|---|
| ID NO. | of Binding | Orientation is 5′-3′ |
| 1 | 1 | CTTCCGGAAA CAAGACCTCC |
| 2 | 21 | TCACCACAGC CTTGCAACAT |
| 3 | 26 | TGCCTTCACC ACAGCCTTGC |
| 4 | 42 | GAGGCTAGGC TGCACCTGCC |
| 5 | 61 | CAGGGTGTAG CTTGAGCAGG |
| 6 | 81 | GGGCCTCATG CGTGGAGGGC |
| 7 | 102 | CACCATCTCC AGAGTTCTGC |
| 8 | 121 | CCTTTTCTGC CCTTGTAGGC |
| 9 | 141 | GACAGCGGCT GCCGACTTGT |
| 10 | 161 | ACCACAGCTG GTGCCCTCAG |
| 11 | 181 | CCTCAGGTCT TGGCTCCTGC |
| 12 | 201 | TTCTAAGAGG ACACTTCCAC |
| 13 | 221 | ACCTTGCTGG GCACTCCCCA |
| 14 | 241 | ATAGCACCAG TAGCGGGTAC |
| 15 | 261 | GGGAGAGATG GGAATTCTGG |
| 16 | 281 | CAGAGCTCAG GCAGAGAGCA |
| 17 | 301 | CCCAGGGAGG AGCTAAGGCC |
| 18 | 319 | CACCTGTCCT CTACCAAGCC |
| 19 | 341 | CCTACATCCC ATGAGGGCCT |
| 20 | 352 | CTCTCAGACA GCCTACATCC |
| 21 | 361 | TCCACTCCCC TCTCAGACAG |
| 22 | 381 | CTCCTTCACC CCTTCCTCTT |
| 23 | 401 | CATAGTCAAA TGGCAGACAG |
| 24 | 421 | CATGAGTCAA AGGCCATTTG |
| 25 | 441 | CAGTGAGGAG GACAGGGTCC |
| 26 | 461 | CCTCCACTCC ACCCTGCCCC |
| 27 | 481 | ATACCAGCCT AGTAGCTCCC |
| 28 | 501 | ATAGAGGAAG TAAGATTTTT |
| 29 | 521 | GGGCAGCAGC GGCTCAGAGA |
| 30 | 541 | CTCGAGGTCC CTTCCCACAG |
| 31 | 561 | ACAGGGAAGG ATGCTTCACA |
| 32 | 581 | GGGCAGACTG GACAGCAGCT |
| 33 | 601 | GCTTCTCCAG AGGGTCTGGC |
| 34 | 621 | ACCCATGCTG GGGGGCAGGG |
| 35 | 641 | TGCAGGGCGC TGCGGCAGAA |
| 36 | 661 | GCACCAGGAG AGACAGCGGG |
| 37 | 681 | CATGGCCAGC ATGATGGCCT |
| 38 | 701 | AAGGTACCCA GGGCCAGGGT |
| 39 | 721 | CACAGGGTAG GAAGGCAGGC |
| 40 | 741 | CAGGCCGTGG GGCTGGAGCT |
| 41 | 760 | ACAGCCAGTT GCAGTTCACC |
| 42 | 773 | ACAGACTTCA GGAACAGCCA |
| 43 | 781 | AGTGGGGCAC AGACTTCAGG |
| 44 | 801 | ACGGGGTGCT GCCATGGAGA |
| 45 | 821 | GAAAGGCTGG TGACATTGCC |
| 46 | 841 | GGATGCGGTT GGAGGACAAG |
| 47 | 861 | GTCAGAATCA TGGAGGTGGT |
| 48 | 881 | AGGCTGGGCA GGTGGGCAAA |
| 49 | 903 | CCACTTGAGG TTGAGATGCC |
| 50 | 921 | GCCAACCGGC GGGCAGTTCC |
| 51 | 941 | GGGAAGTGCA TGGGGCTGAG |
| 52 | 961 | GCTCGATGGT CATGTGGCAG |
| 53 | 981 | CACAGCCAAG AAGGTGCTGG |
| 54 | 1001 | TTTAGCTCTT CCAGGGTGGG |
| 55 | 1009 | AGCTCAGGTT TAGCTCTTCC |
| 56 | 1021 | TGATGTTGTT GTAGCTCAGG |
| 57 | 1041 | GGGCAGCGCA GGCACAGTCA |
| 58 | 1061 | GACAGGGATA TGAGGGATTT |
| 59 | 1081 | GGATGTTGGT ATGGCTGAGG |
| 60 | 1101 | GCTGGCAGAG TCTAGCATCA |
| 61 | 1121 | AGGGCATGCA GGCCGGCGAG |
| 62 | 1141 | CGTCCATGAA TAGGAAGCGC |
| 63 | 1161 | GTTCTTGTAA TAACAGTTGC |
| 64 | 1181 | TCCAGTGCCT GCCTGCAGGG |
| 65 | 1187 | GCCACCTCCA GTGCCTGCCT |
| 66 | 1201 | GGAGGGCACC CGGGGCCACC |
| 67 | 1221 | GGTGAGGTTG CCCAGGCCAA |
| 68 | 1241 | TTGTACTTGA GTGACAGGTG |
| 69 | 1261 | GGGGCACCAC AGTGAGGTTG |
| 70 | 1281 | CAGGCTGGAA GGCAGGTTGC |
| 71 | 1301 | TAGGACAACA GCAGATACTC |
| 72 | 1321 | CCAGTTTGAC GATGCGGTTG |
| 73 | 1341 | ATTGGCCAGG TCCTCAGGCG |
| 74 | 1361 | AGCACACGCA GGGCGGTCAG |
| 75 | 1381 | GGCAATTTCC GCCCACATCG |
| 76 | 1401 | GGGAGCGTGG TCGCAGCGGC |
| 77 | 1421 | GGGCACTCCA TGCAGGGGTT |
| 78 | 1441 | GTAGCTGGGG GAAGTGACGA |
| 79 | 1461 | GTGGCTGAAG GTATCGGGAT |
| 80 | 1481 | AGGCCTTCAA GACGGCTCAG |
| 81 | 1501 | GAGAACTGTC CTTCAACACC |
| 82 | 1521 | ACTGGCATTC AGCCAGGAGA |
| 83 | 1527 | GAACCAACTG GCATTCAGCC |
| 84 | 1541 | TTTCCCAGCC CACGGAACCA |
| 85 | 1561 | TCAGGTCCAG CACTCGGAGG |
| 86 | 1566 | CTCACTCAGG TCCAGCACTC |
| 87 | 1568 | TTCTCACTCA GGTCCAGCAC |
| 88 | 1581 | TTTGTAGAGG AAGTTCTCAC |
| 89 | 1601 | GCCTTGGTTT TAGTGATGCA |
| 90 | 1621 | GCTGTGTTAG GCCCTGGAAG |
| 91 | 1641 | GGACAGGTTA AGCTTGCGCA |
| 92 | 1661 | ACCCTCTTTT GGTAATTGAA |
| 93 | 1681 | GAGACAGGTG GGCAAAGGAC |
| 94 | 1701 | GCTCCCGAAG GAAGGGGCCA |
| 95 | 1721 | AGCTCCTTCA GGGCGACCAG |
| 96 | 1741 | AGAAGATGCC GTGCATGTCC |
| 97 | 1761 | GGTCTCATCG AGTGAGCGGA |
| 98 | 1781 | CGGGCCAGTG GCCGGAGCGT |
| 99 | 1801 | GAGTCTGGAG CATGGGCAGG |
| 100 | 1821 | GAAGTTCATC TGCAGACGCA |
| 101 | 1841 | CCGAGCTGGG CCTGGTTGAT |
| 102 | 1861 | CAGGGAAGGC CCTGAAGATG |
| 103 | 1881 | CAGGTCCACG TAGCGCAGGC |
| 104 | 1901 | CCGCTGATGC GGTTGTCCGA |
| 105 | 1921 | TGGCTGTCAG CTCCGAAGCT |
| 106 | 1941 | TCCATCTGCC TCCCCCATGG |
| 107 | 1961 | TGCAGCCAGA CCTTCTCCCC |
| 108 | 1981 | CCGGAGCAAG GTCCCCAGGC |
| 109 | 2001 | GCTGGGAGTG TCCACTGGGG |
| 110 | 2021 | TTGGGCCTGA AGTCTTCAGA |
| 111 | 2041 | TGAAGTTGAG GGTGCTGCAG |
| 112 | 2061 | GTTCCGTGAC AGATCCAAGG |
| 113 | 2081 | GGCTGCACGG TCACCAGGTT |
| 114 | 2101 | AGAGCTGGGC AAACATCTCC |
| 115 | 2121 | GCGCAGGCAC TGCAGGTGCG |
| 116 | 2139 | GATGCAGTTG TGGCTCAGGC |
| 117 | 2141 | GAGATGCAGT TGTGGCTCAG |
| 118 | 2161 | GGGAGCCATT GACTGCCTGC |
| 119 | 2181 | ACCGGTCAGC GGCAGGAACT |
| 120 | 2201 | GACAGGTCTA GCACCTGCAG |
| 121 | 2221 | AGAGGTCCAG CTTATTGTGG |
| 122 | 2241 | CGTGAATGAG TGCTCGTGGT |
| 123 | 2261 | GCCTCCAGTC GTGGTAGCTC |
| 124 | 2281 | TGTTGTAGCT GAGGTCCAGG |
| 125 | 2284 | GGCTGTTGTA GCTGAGGTCC |
| 126 | 2301 | CTGCATGCCA AAGGGCTGGC |
| 127 | 2321 | CTGAAGTTGT GGCCCACGCC |
| 128 | 2341 | TGCGCAGGTG AGCCACGAAG |
| 129 | 2361 | CAGGCTGAGG TGGCGCAGGG |
| 130 | 2381 | CTGTGGATGT TGTTGTGGGC |
| 131 | 2401 | AGAGCTGCTG GGACACTTGG |
| 132 | 2421 | GGCCCGCAGC GACGTACTGC |
| 133 | 2441 | GCATTGCCGC TGAAGTCCAG |
| 134 | 2461 | CGGCCCACAT ATGGCCCAGT |
| 135 | 2481 | GTGCAGATAG AGGTCTCCCT |
| 136 | 2501 | CCGCTCAGGC CTTGGAAGAA |
| 137 | 2521 | ACAAGTCCAG CCAGATCAAA |
| 138 | 2541 | GGTGTGCAGG CGGTTCTGGG |
| 139 | 2561 | CGCAGGGTTT GGGGCAGGAG |
| 140 | 2581 | GTAGGCTCTT GGGGAGGTTG |
| 141 | 2601 | GTCACGGAGA CGCAGCACCT |
| 142 | 2621 | TTAAAGAAGG CCAGGTAATT |
| 143 | 2641 | GGAAGTGGAG GCTCCACCAC |
| 144 | 2661 | GAGGACTTCC AGTTTGGGCA |
| 145 | 2681 | AGCTGGTTTC CTGCCAGGTC |
| 146 | 2701 | TGCCATTGGT CAGGGCCTTC |
| 147 | 2721 | CCGGGTGCCA GCAGGCAGGC |
| 148 | 2741 | CTGACATCCA GCCTCCGGAG |
| 149 | 2761 | CGAAGCTGAT GCTGTTGCAG |
| 150 | 2781 | GGAAAAGAAG CCGGGGGCCA |
| 151 | 2801 | TCTCGCAGCT CCTTGGCCTT |
| 152 | 2821 | CGTTGGCGCT AAGGTTGAGC |
| 153 | 2841 | GTGGTCCACT GTCTTGAGGG |
| 154 | 2861 | GCCAGGGGCC CAAACCAGGA |
| 155 | 2881 | CTAGTATTTG CAGGGCACTC |
| 156 | 2901 | CAGAGGGTTG GCGCTTACAT |
| 157 | 2921 | GCCGCCCCAC AGGCGCAGTG |
| 158 | 2941 | CCAGCAGGAA GTCCATAAAG |
| 159 | 2947 | GCACCTCCAG CAGGAAGTCC |
| 160 | 2961 | GGGCACGGCA GCCTGCACCT |
| 161 | 2981 | TTCACCCGGC TGGGCAGACC |
| 162 | 3001 | GCTGGCCCGG ACTGCCACAC |
| 163 | 3021 | AAAGATGCTG AGGCCCTGGA |
| 164 | 3041 | CAGAGGCGCA GGTCCTGTGC |
| 165 | 3061 | AGGAGAGGGC CTCATCCAGG |
| 166 | 3081 | CGAGAGGGCG AAACAGTCCC |
| 167 | 3100 | CCAGAGCCAC AGCCAGCAGC |
| 168 | 3121 | GCAGCATGGG CACACCCAGG |
| 169 | 3141 | GTCCCAGCCA CAGAGGTGAT |
| 170 | 3161 | AGGTGGAAGC AGTACCAGAG |
| 171 | 3181 | AGGGAAGCCA GGCCAGGCAC |
| 172 | 3201 | CCCACTTTGC CGCCCCCGCC |
| 173 | 3221 | GGCAGGGCAT CCTCATCTCG |
| 174 | 3241 | AGACCACGAA GGCATCGTAG |
| 175 | 3261 | TGCGCTCTGC GTTTTGTCGA |
| 176 | 3281 | TTGTACACCC AGTCTGCCAC |
| 177 | 3301 | CCAGCTGCCC CCGAAGCTCG |
| 178 | 3321 | CCAGCGCCCA CGGCACTCCT |
| 179 | 3341 | TCCAGGCACA GGCGGAGTGC |
| 180 | 3361 | CAGGCAGCCA GTCGCGTTCC |
| 181 | 3381 | GTTCTCAAAG AGGGTTTTGC |
| 182 | 3401 | CCATAGACCG AGGCCCACAG |
| 183 | 3421 | CAAACAGCGT CTTGCGGCTG |
| 184 | 3441 | CCGGTCCGTG TGGGCCAGCA |
| 185 | 3461 | GCGCGCAAGA GACCACTGAC |
| 186 | 3481 | GCTGGGCCAG CAGGAAGCTG |
| 187 | 3501 | GCGGTCCTCC AGCAGGCGCT |
| 188 | 3521 | ACCAGCACCA CGACGTCCTT |
| 189 | 3530 | CTCAGGATCA CCAGCACCAC |
| 190 | 3541 | GGCCGTCAGG GCTCAGGATC |
| 191 | 3561 | CCGCACATAG CGGGAGCGGC |
| 192 | 3581 | CGGCAGAGGC GCTGGCGCAG |
| 193 | 3601 | GCCAGAGGAG GACACTCTGG |
| 194 | 3621 | CTGACCACTG GGCTGGTGGG |
| 195 | 3641 | AGCTGGGCCC AGAAGCTGCG |
| 196 | 3661 | CCCTGGTCAG GGCCATGCCC |
| 197 | 3681 | GTTATAGAAG TGGTGGTTGT |
| 198 | 3701 | GGTCCCTGGC AGAAGTTCCG |
| 199 | 3721 | CTCACGGCTA TTCGGCCGTG |
| 200 | 3741 | GGCACCGTGC AGGATTCCGG |
| 201 | 3761 | GGTGAGGTGA GTGTGGAGGT |
| 202 | 3781 | GGTCAGACCA GGCAGGCAGA |
| 203 | 3801 | GAGGGAGGCG AGCAGGGGAG |
| 204 | 3821 | CTCTGTGTCA GGTGTGGGGT |
| 205 | 3841 | TAGCATTTAT TGAGTGCCTG |

Underlined nucleotides are 2′-O-methylribonucleotides; all others are 2′-deoxyribonucleotides. All sequences are phosphorothioate backbone modified. In the exemplar antisense oligonucleotides according to the invention, when a “CG” dinucleotide is contained in the sequence, such oligonucleotide is modified to remove or prevent the immune stimulatory properties of the oligonucleotide.

In a second aspect, the invention provides a composition comprising at least one optimized antisense oligonucleotide according to the invention and a physiologically acceptable carrier, diluent or excipient. The characteristics of the carrier will depend on the route of administration. Such a composition may contain, in addition to the synthetic oligonucleotide and carrier, diluents, fillers, salts, buffers, stabilizers, solubilizers, and other materials well known in the art. The pharmaceutical composition of the invention may also contain other active factors and/or agents which enhance inhibition of TLR9 expression. For example, combinations of synthetic oligonucleotides, each of which is directed to different regions of the TLR9 mRNA, may be used in the pharmaceutical compositions of the invention. The pharmaceutical composition of the-invention may further contain nucleotide analogs such as azidothymidine, dideoxycytidine, dideoxyinosine, and the like. Such additional factors and/or agents may be included in the pharmaceutical composition to produce a synergistic, additive or enhanced effect with the synthetic oligonucleotide of the invention, or to minimize side-effects caused by the synthetic oligonucleotide of the invention. The pharmaceutical composition of the invention may be in the form of a liposome in which the synthetic oligonucleotides of the invention is combined, in addition to other pharmaceutically acceptable carriers, with amphipathic agents such as lipids which exist in aggregated form as micelles, insoluble monolayers, liquid crystals, or lamellar layers which are in aqueous solution. Suitable lipids for liposomal formulation include, without limitation, monoglycerides, diglycerides, sulfatides, lysolecithin, phospholipids, saponin, bile acids, and the like. One particularly useful lipid carrier is lipofectin. Preparation of such liposomal formulations is within the level of skill in the art, as disclosed, for example, in U.S. Pat. Nos. 4,235,871; 4,501,728; 4,837,028; and 4,737,323. The pharmaceutical composition of the invention may further include compounds such as cyclodextrins and the like that enhance delivery of oligonucleotides into cells or slow release polymers.

In a third aspect, the invention provides a method of inhibiting TLR9 expression. In this method, an oligonucleotide or multiple oligonucleotides of the invention are specifically contacted or hybridized with TLR9 mRNA either in vitro or in a cell.

In a fourth aspect, the invention provides methods for inhibiting the expression of TLR9 in an animal, particularly a human, such methods comprising administering to the animal a compound or composition according to the invention.

In a fifth aspect, the invention provides a method for inhibiting a TLR-mediated immune response in a vertebrate, the method comprising administering to the vertebrate a TLR9 antisense oligonucleotide according to the invention in a pharmaceutically effective amount, wherein routes of administration include, but are not limited to, parenteral, mucosal delivery, oral, sublingual, transdermal, topical, inhalation, intranasal, aerosol, intraocular, intratracheal, intrarectal, vaginal, by gene gun, dermal patch or in eye drop or mouthwash form.

In a sixth aspect, the invention provides a method for therapeutically treating a vertebrate having a disease mediated by TLR9, such method comprising administering to the vertebrate, particularly a human, a TLR9 antisense oligonucleotide of the invention in a pharmaceutically effective amount.

In certain embodiments, the disease is cancer, an autoimmune disorder, airway inflammation, inflammatory disorders, infectious disease, malaria, Lyme disease, ocular infections, conjunctivitis, skin disorders, psoriasis, scleroderma, cardiovascular disease, atherosclerosis, chronic fatigue syndrome, sarcoidosis, transplant rejection, allergy, asthma or a disease caused by a pathogen. Preferred autoimmune disorders include without limitation lupus erythematosus, multiple sclerosis, type I diabetes mellitus, irritable bowel syndrome, Chron's disease, rheumatoid arthritis, septic shock, alopecia universalis, acute disseminated encephalomyelitis, Addison's disease, ankylosing spondylitis, antiphospholipid antibody syndrome, autoimmune hemolytic anemia, autoimmune hepatitis, Bullous pemphigoid, chagas disease, chronic obstructive pulmonary disease, coeliac disease, dermatomyositis, endometriosis, Goodpasture's syndrome, Graves' disease, Guillain-Barré syndrome, Hashimoto's disease, hidradenitis suppurativa, idiopathic thrombocytopenic purpura, interstitial cystitis, morphea, myasthenia gravis, narcolepsy, neuromyotonia, pemphigus, pernicious anaemia, polymyositis, primary biliary cirrhosis, schizophrenia, Sjögren's syndrome, temporal arteritis (“giant cell arteritis”), vasculitis, vitiligo, vulvodynia and Wegener's granulomatosis. In certain embodiments, inflammatory disorders include without limitation airway inflammation, asthma, autoimmune diseases, chronic inflammation, chronic prostatitis, glomerulonephritis, Behçet's disease, hypersensitivities, inflammatory bowel disease, reperfusion injury, rheumatoid arthritis, transplant rejection, ulcerative colitis, uveitis, conjunctivitis and vasculitis.

In a seventh aspect, the invention provides methods for preventing a disease or disorder in an animal, particularly a human, at risk of contracting or developing a disease or disorder mediated by TLR9. The method according to this aspect comprises administering to the animal a prophylactically effective amount of an antisense oligonucleotide or composition according to the invention. Such diseases and disorders include, without limitation, cancer, an autoimmune disorder, airway inflammation, inflammatory disorders, infectious disease, malaria, Lyme disease, ocular infections, conjunctivitis, skin disorders, psoriasis, scleroderma, cardiovascular disease, atherosclerosis, chronic fatigue syndrome, sarcoidosis, transplant rejection, allergy, asthma or a disease caused by a pathogen in a vertebrate, such method comprising administering to the vertebrate, particularly a human, a TLR9 antisense oligonucleotide of the invention in a pharmaceutically effective amount. Autoimmune disorders include, without limitation, lupus erythematosus, multiple sclerosis, type I diabetes mellitus, irritable bowel syndrome, Chron's disease, rheumatoid arthritis, septic shock, alopecia universalis, acute disseminated encephalomyelitis, Addison's disease, ankylosing spondylitis, antiphospholipid antibody syndrome, autoimmune hemolytic anemia, autoimmune hepatitis, Bullous pemphigoid, chagas disease, chronic obstructive pulmonary disease, coeliac disease, dermatomyositis, endometriosis, Goodpasture's syndrome, Graves' disease, Guillain-Barré syndrome, Hashimoto's disease, hidradenitis suppurativa, idiopathic thrombocytopenic purpura, interstitial cystitis, morphea, myasthenia gravis, narcolepsy, neuromyotonia, pemphigus, pernicious anaemia, polymyositis, primary biliary cirrhosis, schizophrenia, Sjögren's syndrome, temporal arteritis (“giant cell arteritis”), vasculitis, vitiligo, vulvodynia and Wegener's granulomatosis. Inflammatory disorders include, without limitation, airway inflammation, asthma, autoimmune diseases, chronic inflammation, chronic prostatitis, glomerulonephritis, Behçet's disease, hypersensitivities, inflammatory bowel disease, reperfusion injury, rheumatoid arthritis, transplant rejection, ulcerative colitis, uveitis, conjunctivitis and vasculitis.

In an eighth aspect of the invention, the invention provides methods for down-regulating TLR9 expression and thus preventing the “off-target” activity of certain other antisense molecules, or other compounds or drugs that have a side effect of activating TLR9. Certain antisense and other DNA and/or RNA-based compounds that are designed to down-regulate expression of targets other than TLR9 also are recognized by TLR9 proteins and induce an immune response. This activity can be referred to as “off-target” effects. The TLR9 antisense oligonucleotides according to the invention have the ability to down-regulate TLR9 expression and thus prevent the TLR9-mediated off-target activity of the non-TLR9 targeted antisense molecules. For example, the TLR9 antisense oligonucleotide according to the invention can be administered in combination with one or more antisense oligonucleotides, which are not the same target as the antisense molecule of the invention, and which comprise an immunostimulatory motif that would activate a TLR9-mediate immune response but for the presence the TLR9 antisense oligonucleotide according to the invention. Thus, for example, the TLR9 antisense oligonucleotide may be administered in combination with one or more antisense oligonucleotides or RNAi molecules (for example: siRNA, miRNA, ddRNA and eiRNA), which are not targeted to the same molecule as the antisense oligonucleotides of the invention.

In the various methods according to the invention, a therapeutically or prophylactically effective amount of a synthetic oligonucleotide of the invention and effective in inhibiting the expression of TLR9 is administered to a cell. This cell may be part of a cell culture, a neovascularized tissue culture, or may be part or the whole body of an animal such as a human or other mammal. Administration may be by any suitable route, including, without limitation, parenteral, mucosal delivery, oral, sublingual, transdermal, topical, inhalation, intranasal, aerosol, intraocular, intratracheal, intrarectal, vaginal, by gene gun, dermal patch or in eye drop or mouthwash form. Administration of the therapeutic compositions of TLR9 antisense oligonucleotide can be carried out using known procedures at dosages and for periods of time effective to reduce symptoms or surrogate markers of the disease, depending on the condition and response, as determined by those with skill in the art. It may be desirable to administer simultaneously, or sequentially a therapeutically effective amount of one or more of the therapeutic TLR9 antisense oligonucleotides of the invention to an individual as a single treatment episode. In some exemplar embodiments of the methods of the invention described above, the oligonucleotide is administered locally and/or systemically. The term “administered locally” refers to delivery to a defined area or region of the body, while the term “systemic administration” is meant to encompass delivery to the whole organism.

In any of the methods according to the invention, the TLR9 antisense oligonucleotide can be administered in combination with any other agent useful for treating the disease or condition that does not diminish the immune modulatory effect of the TLR9 antisense oligonucleotide. In any of the methods according to the invention, the agent useful for treating the disease or condition includes, but is not limited to, one or more vaccines, antigens, antibodies, cytotoxic agents, allergens, antibiotics, antisense oligonucleotides, TLR agonist, TLR antagonist, siRNA, miRNA, peptides, proteins, gene therapy vectors, DNA vaccines, adjuvants or kinase inhibitors to enhance the specificity or magnitude of the immune response, or co-stimulatory molecules such as cytokines, chemokines, protein ligands, trans-activating factors, peptides and peptides comprising modified amino acids. For example, in the treatment of autoimmune disease, it is contemplated that the TLR9 antisense oligonucleotide may be administered in combination with one or more targeted therapeutic agents and/or monoclonal antibodies. Alternatively, the agent can include DNA vectors encoding for antigen or allergen. In these embodiments, the TLR9 antisense oligonucleotide of the invention can produce direct immune modulatory or suppressive effects. When co-administered with one or more other therapies, the synthetic oligonucleotide of the invention may be administered either simultaneously with the other treatment(s), or sequentially.

In the various methods according to the invention the route of administration may be, without limitation, parenteral, mucosal delivery, oral, sublingual, transdermal, topical, inhalation, intranasal, aerosol, intraocular, intratracheal, intrarectal, vaginal, by gene gun, dermal patch or in eye drop or mouthwash form.

When a therapeutically effective amount of synthetic oligonucleotide of the invention is administered orally, the synthetic oligonucleotide will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% synthetic oligonucleotide and preferably from about 25 to 90% synthetic oligonucleotide. When administered in liquid form, a liquid carrier such as water, petroleum, oils of animal or plant origin such as peanut oil, mineral oil, soybean oil, sesame oil, or synthetic oils may be added. The liquid form of the pharmaceutical composition may further contain physiological saline solution, dextrose or other saccharide solution or glycols such as ethylene glycol, propylene glycol or polyethylene glycol. When administered in liquid form, the pharmaceutical composition contains from about 0.5 to 90% by weight of the synthetic oligonucleotide or from about 1 to 50% synthetic oligonucleotide.

When a therapeutically effective amount of synthetic oligonucleotide of the invention is administered by parenteral, mucosal delivery, oral, sublingual, transdermal, topical, inhalation, intranasal, aerosol, intraocular, intratracheal, intrarectal, vaginal, by gene gun, dermal patch or in eye drop or mouthwash form, the synthetic antisense oligonucleotide will be in the form of a pyrogen-free, parenterally acceptable aqueous solution. The preparation of such parenterally acceptable solutions, having due regard to pH, isotonicity, stability, and the like, is within the skill in the art. An exemplar pharmaceutical composition for parenteral, mucosal delivery, oral, sublingual, transdermal, topical, inhalation, intranasal, aerosol, intraocular, intratracheal, intrarectal, vaginal, by gene gun, dermal patch or in eye drop or mouthwash form should contain, in addition to the synthetic oligonucleotide, an isotonic vehicle such as Sodium Chloride Injection, Ringer's Injection, Dextrose Injection, Dextrose and Sodium Chloride Injection, Lactated Ringer's Injection or other vehicle as known in the art. The pharmaceutical composition of the present invention may also contain stabilizers, preservatives, buffers, antioxidants or other additives known to those of skill in the art.

When administered parenteral, mucosal delivery, oral, sublingual, transdermal, topical, inhalation, intranasal, aerosol, intraocular, intratracheal, intrarectal, vaginal, by gene gun, dermal patch or in eye drop or mouthwash form, doses ranging from 0.01% to 10% (weight/volume) may be used. When administered in liquid form, a liquid carrier such as water, petroleum, oils of animal or plant origin such as peanut oil, mineral oil, soybean oil, sesame oil or synthetic oils may be added. Topical administration may be by liposome or transdermal time-release patch.

The amount of synthetic oligonucleotide in the pharmaceutical composition of the present invention will depend upon the nature and severity of the condition being treated, and on the nature of prior treatments which the patent has undergone. It is contemplated that the various pharmaceutical compositions used to practice the method of the present invention should contain about 10 micrograms to about 20 mg of synthetic oligonucleotide per kg body or organ weight.

The duration of intravenous therapy using the pharmaceutical composition of the present invention will vary, depending on the severity of the disease being treated and the condition and potential idiosyncratic response of each individual patient.

Some diseases lend themselves to acute treatment while others require longer term therapy. Both acute and long term intervention in diseases are worthy goals. Injections of antisense oligonucleotides against TLR9 can be an effective means of inhibiting certain diseases in an acute situation. However for long term therapy over a period of weeks, months or years, systemic delivery (intraperitoneal, intramuscular, subcutaneous, intravenous) either with carriers such as saline, slow release polymers or liposomes are likely to be considered.

In some chronic diseases, systemic administration of oligonucleotides may be preferable. The frequency of injections is from continuous infusion to once a month, several times per month or less frequently will be determined based on the disease process and the biological half life of the oligonucleotides.

The oligonucleotides and methods of the invention are also useful for examining the function of the TLR9 gene in a cell or in a control mammal or in a mammal afflicted with a disease associated with TLR9 or immune stimulation through TLR9. In such use, the cell or mammal is administered the oligonucleotide, and the expression of TLR9 mRNA or protein is examined.

Without being limited to any theory or mechanism, it is generally believed that the activity of oligonucleotides according to the invention depends on the hybridization of the oligonucleotide to the target nucleic acid (e.g. to at least a portion of a genomic region, gene or mRNA transcript thereof), thus disrupting the function of the target. Such hybridization under physiological conditions is measured as a practical matter by observing interference with the function of the nucleic acid sequence. Thus, an exemplar oligonucleotide used in accordance with the invention is capable of forming a stable duplex (or triplex in the Hoogsteen or other hydrogen bond pairing mechanism) with the target nucleic acid; activating RNase H or other in vivo enzymes thereby causing effective destruction of the target RNA molecule; and is capable of resisting nucleolytic degradation (e.g. endonuclease and exonuclease activity) in vivo. A number of the modifications to oligonucleotides described above and others which are known in the art specifically and successfully address each of these exemplar characteristics.

In the various methods of treatment or use of the present invention, a therapeutically or prophylactically effective amount of one, two or more of the synthetic oligonucleotides of the invention is administered to a subject afflicted with or at risk of developing a disease or disorder. The antisense oligonucleotide(s) of the invention may be administered in accordance with the method of the invention either alone or in combination with other known therapies, including but not limited to, one or more vaccines, antigens, antibodies, cytotoxic agents, allergens, antibiotics, antisense oligonucleotides, TLR agonist, TLR antagonist, siRNA, miRNA, peptides, proteins, gene therapy vectors, DNA vaccines, adjuvants or kinase inhibitors to enhance the specificity or magnitude of the immune response, or co-stimulatory molecules such as cytokines, chemokines, protein ligands, trans-activating factors, peptides and peptides comprising modified amino acids. When co-administered with one or more other therapies, the synthetic oligonucleotide of the invention may be administered either simultaneously with the other treatment(s), or sequentially.

The following examples illustrate the exemplar modes of making and practicing the present invention, but are not meant to limit the scope of the invention since alternative methods may be utilized to obtain similar results.

### Example 1

### Preparation of TLR9-Specific Antisense Oligonucleotides

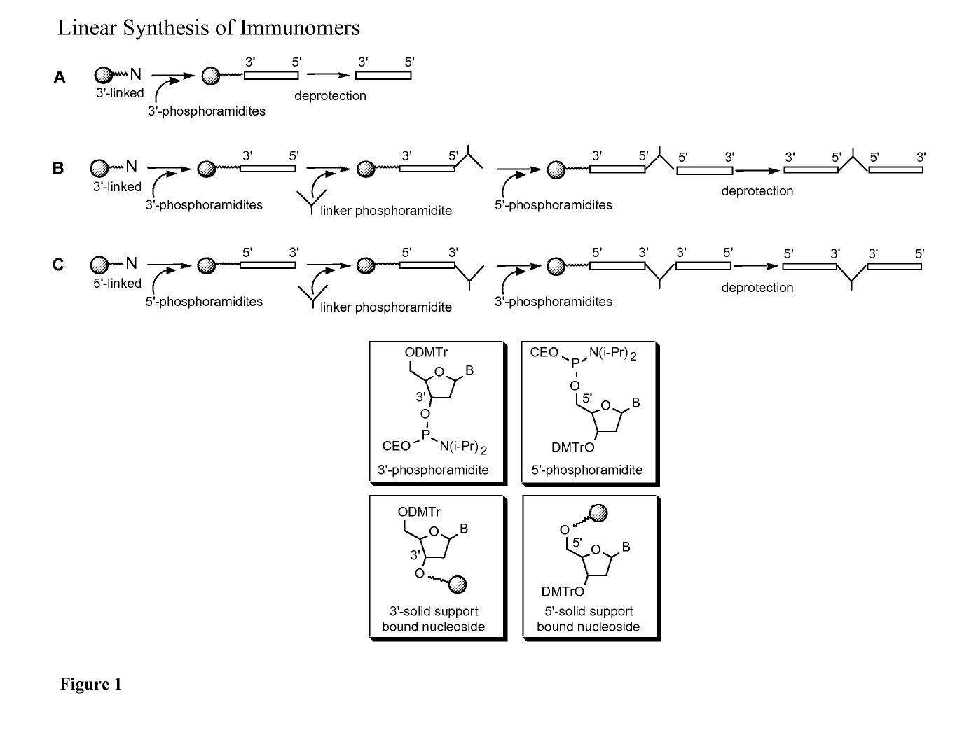

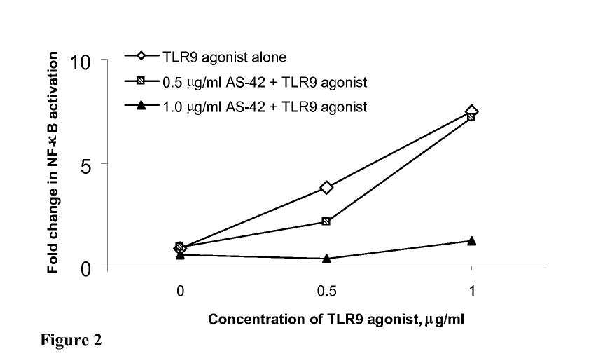

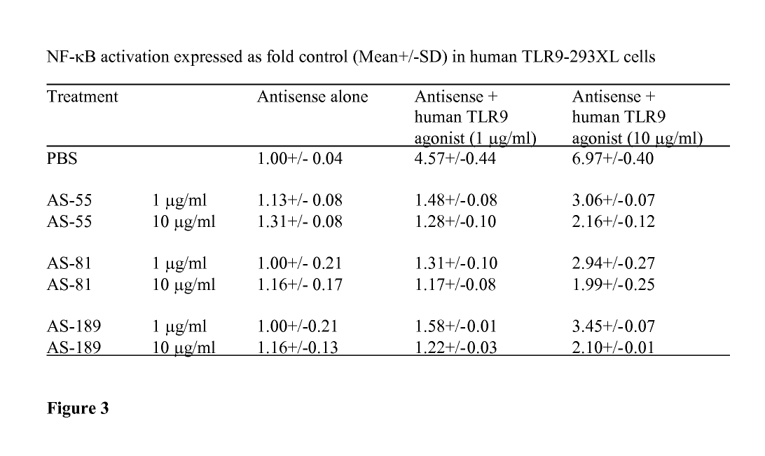

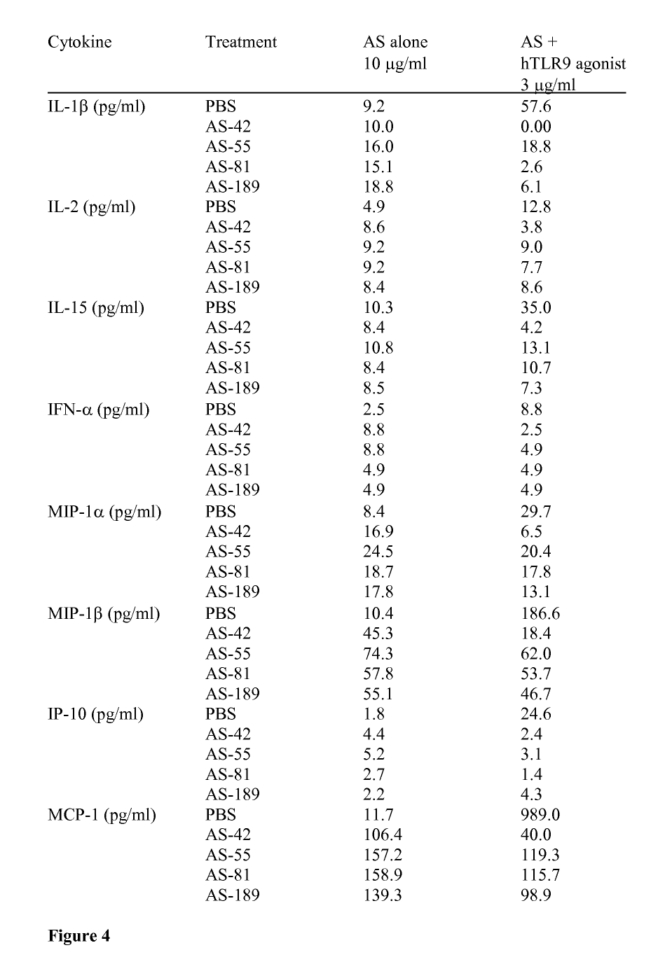

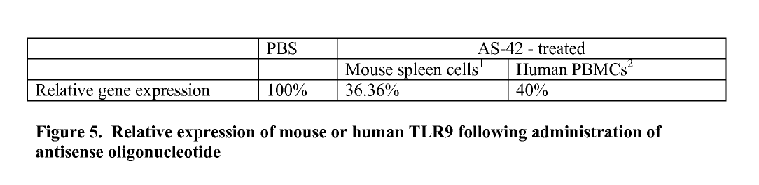

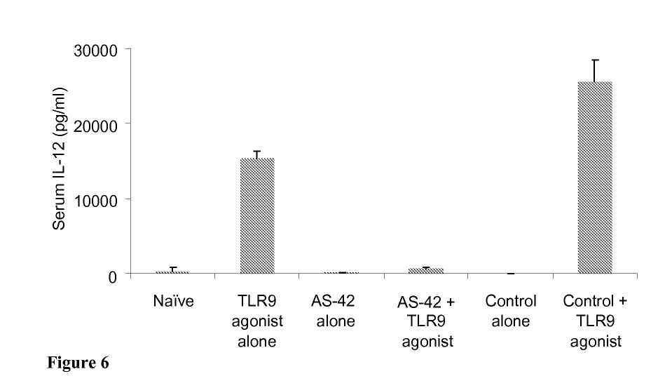

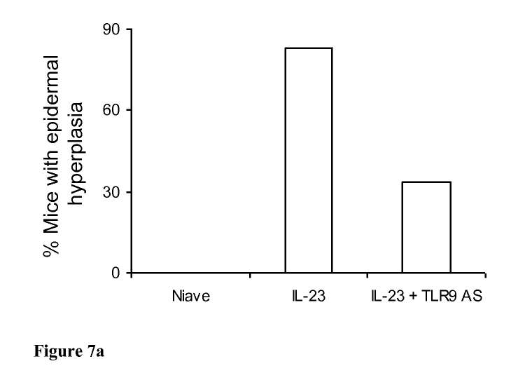

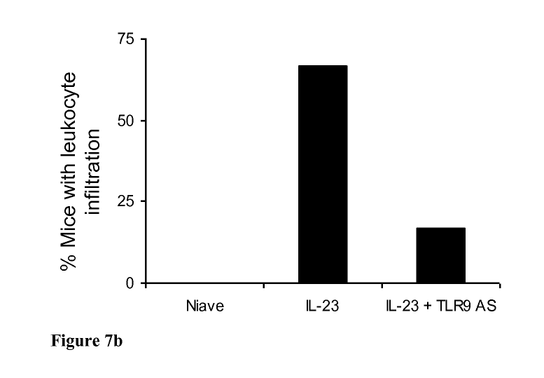

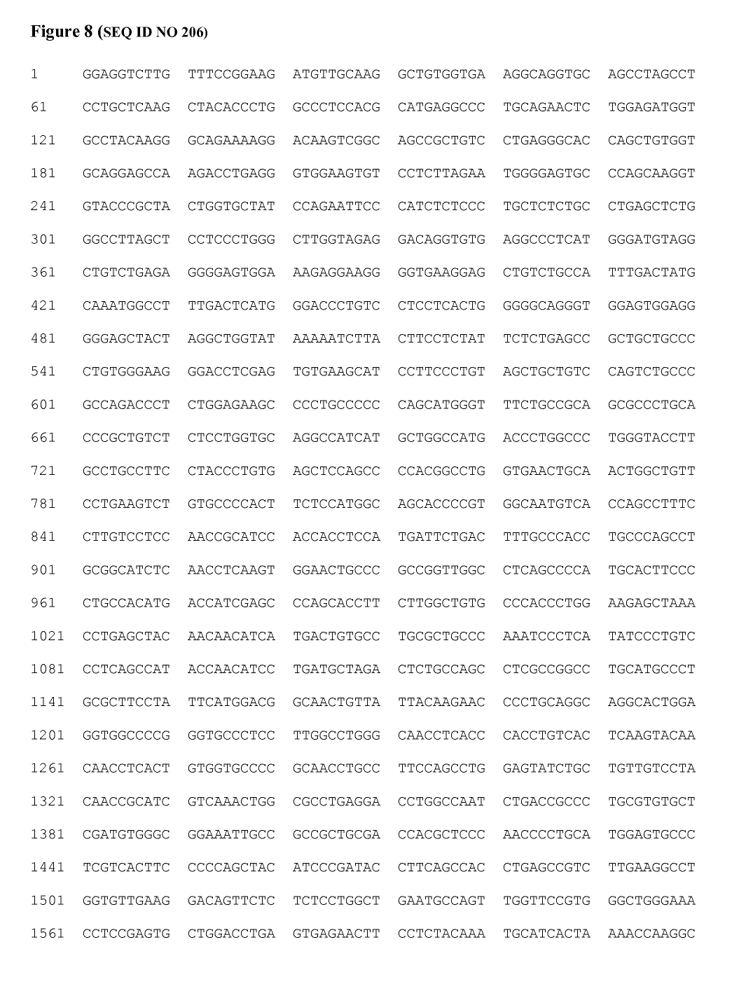

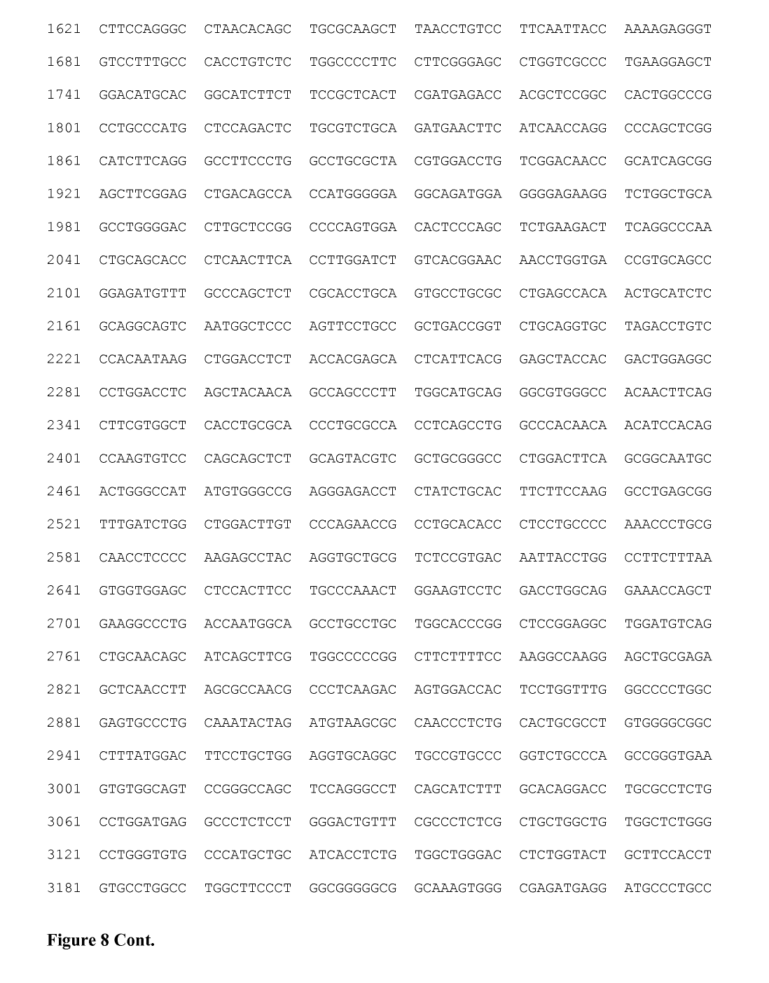

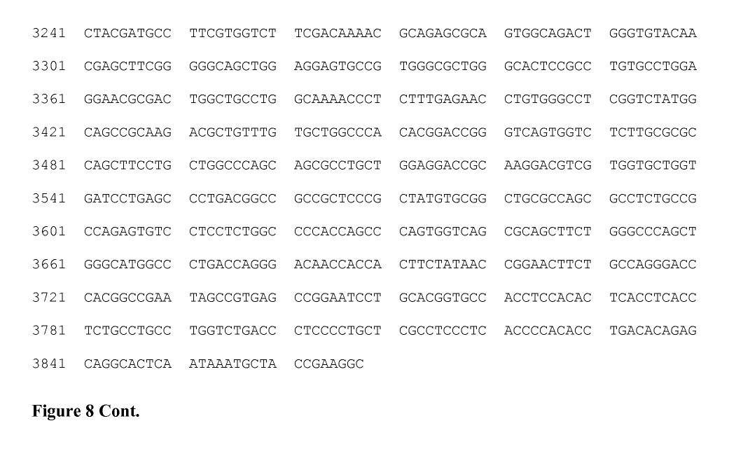

Chemical entities according to the invention were synthesized on a 1 μmol to 0.1 mM scale using an automated DNA synthesizer (OligoPilot II, AKTA, (Amersham) and/or Expedite 8909 (Applied Biosystem)), following the linear synthesis procedure outlined in FIG. 1.

5′-DMT dA, dG, dC and T phosphoramidites were purchased from Proligo (Boulder, Colo.). 5′-DMT 7-deaza-dG and araG phosphoramidites were obtained from Chemgenes (Wilmington, Mass.). DiDMT-glycerol linker solid support was obtained from Chemgenes. 1-(2′-deoxy-β-D-ribofuranosyl)-2-oxo-7-deaza-8-methyl-purine amidite was obtained from Glen Research (Sterling, Va.), 2′-O-methylribonuncleoside amidites were obtained from Promega (Obispo, Calif.). All compounds according to the invention were phosphorothioate backbone modified.

All nucleoside phosphoramidites were characterized by ³¹P and ¹H NMR spectra. Modified nucleosides were incorporated at specific sites using normal coupling cycles recommended by the supplier. After synthesis, compounds were deprotected using concentrated ammonium hydroxide and purified by reverse phase HPLC, detritylation, followed by dialysis. Purified compounds as sodium salt form were lyophilized prior to use. Purity was tested by CGE and MALDI-TOF MS. Endotoxin levels were determined by LAL test and were below 1.0 EU/mg.

### Example 2

### Cell Culture Conditions and Reagents

### HEK293 Cell Culture Assays for TLR9 Antisense Activity

HEK293 or 293 XL cells stably expressing mouse TLR9 (Invivogen, San Diego, Calif.) and human TLR9 respectively, were plated in 48-well plates in 250 μL/well DMEM supplemented with 10% heat-inactivated FBS in a 5% CO2 incubator. At 80% confluence, cultures were transiently transfected with 400 ng/mL of the secreted form of human embryonic alkaline phosphatase (SEAP) reporter plasmid (pNifty2-Seap) (Invivogen) in the presence of 4 μL/mL of lipofectamine (Invitrogen, Carlsbad, Calif.) in culture medium. Plasmid DNA and lipofectamine were diluted separately in serum-free medium and incubated at room temperature for 5 min. After incubation, the diluted DNA and lipofectamine were mixed and the mixtures were incubated further at room temperature for 20 min. Aliquots of 25 μL of the DNA/lipofectamine mixture containing 100 ng of plasmid DNA and 1 μL of lipofectamine were added to each well of the cell culture plate, and the cells were transfected for 6 h. After transfection, medium was replaced with fresh culture medium (no antibiotics), antisense compounds were added to the wells, and incubation continued for 18-20 h. Cells were then stimulated with the TLR9 agonist for 6 h.

At the end of the treatment, 20 μL of culture supernatant was taken from each well and assayed for SEAP assay by the Quanti Blue method according to the manufacturer's protocol (Invivogen). The data are shown as fold increase in NF-κB activity over PBS control.

### Example 3

### Human PBMC Isolation and Determination of Antisense Activity

Peripheral blood mononuclear cells (PBMCs) from freshly drawn healthy volunteer blood (RBC, Brighton, Mass.) were isolated by Ficoll density gradient centrifugation method.

A total of 1×10⁶ PBMCs/200 μl were stimulated with antisense compounds overnight (˜20 hrs) and then stimulated with the TLR agonist for 6 hours. Supernatants were harvested and stored frozen at −20° C. until assayed for cytokines using the human 25-plex AB kit (Invitrogen).

### Example 4

### Mouse Spleen and Human PBMC TLR9 Gene Expression Analysis by Real-Time PCR

Female C57BL/6 mice of 5-6 weeks age (N=3/group) were injected with exemplar TLR9 antisense oligonucleotides according to the invention at 5 mg/kg, or PBS, subcutaneously once a day for five days. 72 hours after the last injections of the exemplar TLR9 antisense oligonucleotides, spleens were collected and total RNA was isolated from spleen cells.

Peripheral blood mononuclear cells (PBMCs) from freshly drawn healthy volunteer blood (RBC, Brighton, Mass.) were isolated by Ficoll density gradient centrifugation method. A total of 1×10⁶ PBMCs/200 μl were stimulated with antisense compounds overnight (˜20 hrs) and total RNA was isolated from PBMCs.

Five hundred ng of total RNA isolated from mouse spleen cells and human PBMCs was used for cDNA synthesis using the High-Capacity cDNA Reverse Transcription Kit (Applied Biosystems) according to the manufacturer's recommendation. Real-time PCR was carried out using 2 μl of cDNA sample for each reaction on StepOnePlus™ Real-time PCR system (Applied Biosystems). Mouse or human TLR9 specific TaqMan gene expression assay primer-probe sets obtained from Applied Biosystems. Mouse or human GAPDH gene was used as housekeeping internal control. The data were analyzed by StepOne software version 2.0 and the results are expressed as change in relative expression compared with PBS control.

### Example 5

### In Vivo Activity of TLR9 Antisense Oligonucleotide

Female C57BL/6 mice of 5-6 weeks age (N=3/group) were injected with exemplar TLR9 antisense oligonucleotides according to the invention at 5 mg/kg, or PBS, subcutaneously once a day for three days. Subsequent to administration of the TLR9 antisense oligonucleotide, mice were injected with 0.25 mg/kg of a TLR9 agonist subcutaneously. Two hours after administration of the TLR9 agonist, blood was collected and IL-12 concentration was determined by ELISA.

### Example 6

### In Vivo Activity of TLR9 Antisense Oligonucleotide in Psoriasis-Like Skin Lesions

Psoriasis-like skin lesions were induced in two groups of 6 week old, female C57Bl/6 mice (n=3) by intradermal injection with 1 μg recombinant mouse IL-23 in 50 μl PBS on days 0, 1, 2, and 3 (total 4 doses). One group of IL-23 injected mice were treated with subcutaneous injections of 200 μg (10 mg/kg body weight) of exemplar TLR9 antisense oligonucleotide (AS) in 100 μl PBS on days −1, 0, and 2 (total 3 doses). For control, one group, of mice were injected with PBS only at the same times as IL-23 and TLR9 AS injection. All mice were euthanized on day 4, and two skin samples were taken from each mouse at IL-23 injection sites for histological examination.

### EQUIVALENTS

Those skilled in the art will recognize, or be able to ascertain, using no more than routine experimentation, numerous equivalents to the specific substances and procedures described herein. For example, antisense oligonucleotides that overlap with the oligonucleotides may be used. Such equivalents are considered to be within the scope of this invention, and are covered by the following claims.

1. A synthetic antisense oligonucleotide 20 to 50 nucleotides in length targeted to TLR9 mRNA (SEQ ID NO: 206), wherein the antisense oligonucleotide has a sequence comprising SEQ ID NOs: 3, 4, 7, 18, 41, 42, 49, 55, 65, 81, 83, 87, 116, 125, 159, 167 or 189, and wherein the oligonucleotide specifically hybridizes to and inhibits the expression of human TLR9.

2-5. (canceled)

6. A composition comprising a synthetic antisense oligonucleotide according to claim 1 and a physiologically acceptable carrier.

7. A method for inhibiting the expression of TLR9, the method comprising administering a synthetic antisense oligonucleotide according to claim 1.

8. A method for inhibiting the expression of TLR9, the method comprising administering a composition according to claim 6.

9. A method for inhibiting the expression of TLR9 in an mammal, the method comprising administering to the mammal a synthetic antisense oligonucleotide according to claim 1.

10. A method for inhibiting the expression of TLR9 in a mammal, the method comprising administering to the mammal a composition according to claim 6.

11. A method for inhibiting a TLR9-mediated immune response in a mammal, the method comprising administering to the mammal a synthetic antisense oligonucleotide according to claim 1 in a pharmaceutically effective amount.

12. A method for inhibiting a TLR9-mediated immune response in a mammal, the method comprising administering to the mammal a composition according to claim 6 in a pharmaceutically effective amount.

13. A method for therapeutically treating a mammal having one or more diseases mediated by TLR9, the method comprising administering to the mammal a synthetic antisense oligonucleotide according to claim 1 in a pharmaceutically effective amount.

14. A method for therapeutically treating a mammal having one or more diseases mediated by TLR9, the method comprising administering to the mammal a composition according to claim 6 in a pharmaceutically effective amount.

15. A method for preventing in a mammal one or more diseases or disorders mediated by TLR9, the method comprising administering to the mammal a synthetic antisense oligonucleotide according to claim 1 in a prophylactically effective amount.

16. A method for preventing in a mammal one or more diseases or disorders mediated by TLR9, the method comprising administering to the mammal a composition according to claim 6 in a prophylactically effective amount.

17. A method for down-regulating TLR9 expression and thus preventing undesired TLR9-mediated immune stimulation by a compound that activates TLR9, the method comprising administering a synthetic antisense oligonucleotide according to claim 1 in combination with one or more compounds which comprise an immunostimulatory motif that would activate a TLR9-mediated immune response but for the presence of the antisense oligonucleotide.

18. A method for down-regulating TLR9 expression and thus preventing undesired TLR9-mediated immune stimulation by a compound that activates TLR9, the method comprising administering a composition according to claim 6 in combination with one or more compounds which comprise an immunostimulatory motif that would activate a TLR9-mediated immune response but for the presence of the composition.

19. The method according to claim 9, wherein the mammal is a human.

20. The method according to claim 13, wherein the one or more diseases are selected from the group consisting of cancer, an autoimmune disorder, airway inflammation, inflammatory disorders, infectious disease, malaria, Lyme disease, ocular infections, conjunctivitis, skin disorders, psoriasis, scleroderma, cardiovascular disease, atherosclerosis, chronic fatigue syndrome, sarcoidosis, transplant rejection, allergy, asthma and a disease caused by a pathogen.

21. The method according to claim 20, wherein the autoimmune disorder is selected from the group consisting of lupus erythematosus, multiple sclerosis, type I diabetes mellitus, irritable bowel syndrome, Chron's disease, rheumatoid arthritis, septic shock, alopecia universalis, acute disseminated encephalomyelitis, Addison's disease, ankylosing spondylitis, antiphospholipid antibody syndrome, autoimmune hemolytic anemia, autoimmune hepatitis, Bullous pemphigoid, chagas disease, chronic obstructive pulmonary disease, coeliac disease, dermatomyositis, endometriosis, Goodpasture's syndrome, Graves' disease, Guillain-Barré syndrome, Hashimoto's disease, hidradenitis suppurativa, idiopathic thrombocytopenic purpura, interstitial cystitis, morphea, myasthenia gravis, narcolepsy, neuromyotonia, pemphigus, pernicious anaemia, polymyositis, primary biliary cirrhosis, schizophrenia, Sjögren's syndrome, temporal arteritis (“giant cell arteritis”), vasculitis, vitiligo, vulvodynia and Wegener's granulomatosis.

22. The method according to claim 20, wherein the inflammatory disorder is selected from the group consisting of airway inflammation, asthma, autoimmune diseases, chronic inflammation, chronic prostatitis, glomerulonephritis, Behçet's disease, hypersensitivities, inflammatory bowel disease, reperfusion injury, rheumatoid arthritis, transplant rejection, ulcerative colitis, uveitis, conjunctivitis and vasculitis.

23. The method according to claim 17, wherein the compound is one or more non-TLR9 antisense oligonucleotides comprising an immunostimulatory motif that would otherwise activate a TLR9-mediated immune response.

24. The method according to claim 7, wherein the route of administration is selected from the group consisting of parenteral, intramuscular, subcutaneous, intraperitoneal, intravenous, mucosal delivery, oral, sublingual, transdermal, topical, inhalation, intranasal, aerosol, intraocular, intratracheal, intrarectal, vaginal, gene gun, dermal patch, eye drop and mouthwash.

25. The method according to claim 7 comprising further administering one or more vaccines, antigens, antibodies, cytotoxic agents, allergens, antibiotics, antisense oligonucleotides, TLR agonist, TLR antagonist, siRNA, miRNA, antisense oligonucleotides, aptamers, proteins, gene therapy vectors, DNA vaccines, adjuvants, co-stimulatory molecules or combinations thereof.

26. The method according to claim 15, wherein the one or more diseases are selected from the group consisting of cancer, an autoimmune disorder, airway inflammation, inflammatory disorders, infectious disease, malaria, Lyme disease, ocular infections, conjunctivitis, skin disorders, psoriasis, scleroderma, cardiovascular disease, atherosclerosis, chronic fatigue syndrome, sarcoidosis, transplant rejection, allergy, asthma and a disease caused by a pathogen.

27. The method according to claim 26, wherein the autoimmune disorder is selected from a group consisting of lupus erythematosus, multiple sclerosis, type I diabetes mellitus, irritable bowel syndrome, Chron's disease, rheumatoid arthritis, septic shock, alopecia universalis, acute disseminated encephalomyelitis, Addison's disease, ankylosing spondylitis, antiphospholipid antibody syndrome, autoimmune hemolytic anemia, autoimmune hepatitis, Bullous pemphigoid, chagas disease, chronic obstructive pulmonary disease, coeliac disease, dermatomyositis, endometriosis, Goodpasture's syndrome, Graves' disease, Guillain-Barré syndrome, Hashimoto's disease, hidradenitis suppurativa, idiopathic thrombocytopenic purpura, interstitial cystitis, morphea, myasthenia gravis, narcolepsy, neuromyotonia, pemphigus, pernicious anaemia, polymyositis, primary biliary cirrhosis, schizophrenia, Sjögren's syndrome, temporal arteritis (“giant cell arteritis”), vasculitis, vitiligo, vulvodynia and Wegener's granulomatosis.

28. The method according to claim 26, wherein the inflammatory disorder is selected from a group consisting of airway inflammation, asthma, autoimmune diseases, chronic inflammation, chronic prostatitis, glomerulonephritis, Behçet's disease, hypersensitivities, inflammatory bowel disease, reperfusion injury, rheumatoid arthritis, transplant rejection, ulcerative colitis, uveitis, conjunctivitis and vasculitis.


SEQUENCE LISTING (206 sequences)

SEQ ID NO: 1 - DNA, 20 bp, Artificial Sequence
  Antisense Oligonucleotide
cttccggaaacaagacctcc

SEQ ID NO: 2 - DNA, 20 bp, Artificial Sequence
  Antisense Oligonucleotide
tcaccacagccttgcaacat

SEQ ID NO: 3 - DNA, 20 bp, Artificial Sequence
  Antisense Oligonucleotide
tgccttcaccacagccttgc

SEQ ID NO: 4 - DNA, 20 bp, Artificial Sequence
  Antisense Oligonucleotide
gaggctaggctgcacctgcc

SEQ ID NO: 5 - DNA, 20 bp, Artificial Sequence
  Antisense Oligonucleotide
cagggtgtagcttgagcagg

SEQ ID NO: 6 - DNA, 20 bp, Artificial Sequence
  Antisense Oligonucleotide
gggcctcatgcgtggagggc

SEQ ID NO: 7 - DNA, 20 bp, Artificial Sequence
  Antisense Oligonucleotide
caccatctccagagttctgc

SEQ ID NO: 8 - DNA, 20 bp, Artificial Sequence
  Antisense Oligonucleotide
ccttttctgcccttgtaggc

SEQ ID NO: 9 - DNA, 20 bp, Artificial Sequence
  Antisense Oligonucleotide
gacagcggctgccgacttgt

SEQ ID NO: 10 - DNA, 20 bp, Artificial Sequence
  Antisense Oligonucleotide
accacagctggtgccctcag

SEQ ID NO: 11 - DNA, 20 bp, Artificial Sequence
  Antisense Oligonucleotide
cctcaggtcttggctcctgc

SEQ ID NO: 12 - DNA, 20 bp, Artificial Sequence
  Antisense Oligonucleotide
ttctaagaggacacttccac

SEQ ID NO: 13 - DNA, 20 bp, Artificial Sequence
  Antisense Oligonucleotide
accttgctgggcactcccca

SEQ ID NO: 14 - DNA, 20 bp, Artificial Sequence
  Antisense Oligonucleotide
atagcaccagtagcgggtac

SEQ ID NO: 15 - DNA, 20 bp, Artificial Sequence
  Antisense Oligonucleotide
gggagagatgggaattctgg

SEQ ID NO: 16 - DNA, 20 bp, Artificial Sequence
  Antisense Oligonucleotide
cagagctcaggcagagagca

SEQ ID NO: 17 - DNA, 20 bp, Artificial Sequence
  Antisense Oligonucleotide
cccagggaggagctaaggcc

SEQ ID NO: 18 - DNA, 20 bp, Artificial Sequence
  Antisense Oligonucleotide
cacctgtcctctaccaagcc

SEQ ID NO: 19 - DNA, 20 bp, Artificial Sequence
  Antisense Oligonucleotide
cctacatcccatgagggcct

SEQ ID NO: 20 - DNA, 20 bp, Artificial Sequence
  Antisense Oligonucleotide
ctctcagacagcctacatcc

SEQ ID NO: 21 - DNA, 20 bp, Artificial Sequence
  Antisense Oligonucleotide
tccactcccctctcagacag

SEQ ID NO: 22 - DNA, 20 bp, Artificial Sequence
  Antisense Oligonucleotide
ctccttcaccccttcctctt

SEQ ID NO: 23 - DNA, 20 bp, Artificial Sequence
  Antisense Oligonucleotide
catagtcaaatggcagacag

SEQ ID NO: 24 - DNA, 20 bp, Artificial Sequence
  Antisense Oligonucleotide
catgagtcaaaggccatttg

SEQ ID NO: 25 - DNA, 20 bp, Artificial Sequence
  Antisense Oligonucleotide
cagtgaggaggacagggtcc

SEQ ID NO: 26 - DNA, 20 bp, Artificial Sequence
  Antisense Oligonucleotide
cctccactccaccctgcccc

SEQ ID NO: 27 - DNA, 20 bp, Artificial Sequence
  Antisense Oligonucleotide
ataccagcctagtagctccc

SEQ ID NO: 28 - DNA, 20 bp, Artificial Sequence
  Antisense Oligonucleotide
atagaggaagtaagattttt

SEQ ID NO: 29 - DNA, 20 bp, Artificial Sequence
  Antisense Oligonucleotide
gggcagcagcggctcagaga

SEQ ID NO: 30 - DNA, 20 bp, Artificial Sequence
  Antisense Oligonucleotide
ctcgaggtcccttcccacag

SEQ ID NO: 31 - DNA, 20 bp, Artificial Sequence
  Antisense Oligonucleotide
acagggaaggatgcttcaca

SEQ ID NO: 32 - DNA, 20 bp, Artificial Sequence
  Antisense Oligonucleotide
gggcagactggacagcagct

SEQ ID NO: 33 - DNA, 20 bp, Artificial Sequence
  Antisense Oligonucleotide
gcttctccagagggtctggc

SEQ ID NO: 34 - DNA, 20 bp, Artificial Sequence
  Antisense Oligonucleotide
acccatgctggggggcaggg

SEQ ID NO: 35 - DNA, 20 bp, Artificial Sequence
  Antisense Oligonucleotide
tgcagggcgctgcggcagaa

SEQ ID NO: 36 - DNA, 20 bp, Artificial Sequence
  Antisense Oligonucleotide
gcaccaggagagacagcggg

SEQ ID NO: 37 - DNA, 20 bp, Artificial Sequence
  Antisense Oligonucleotide
catggccagcatgatggcct

SEQ ID NO: 38 - DNA, 20 bp, Artificial Sequence
  Antisense Oligonucleotide
aaggtacccagggccagggt

SEQ ID NO: 39 - DNA, 20 bp, Artificial Sequence
  Antisense Oligonucleotide
cacagggtaggaaggcaggc

SEQ ID NO: 40 - DNA, 20 bp, Artificial Sequence
  Antisense Oligonucleotide
caggccgtggggctggagct

SEQ ID NO: 41 - DNA, 20 bp, Artificial Sequence
  Antisense Oligonucleotide
acagccagttgcagttcacc

SEQ ID NO: 42 - DNA, 20 bp, Artificial Sequence
  Antisense Oligonucleotide
acagacttcaggaacagcca

SEQ ID NO: 43 - DNA, 20 bp, Artificial Sequence
  Antisense Oligonucleotide
agtggggcacagacttcagg

SEQ ID NO: 44 - DNA, 20 bp, Artificial Sequence
  Antisense Oligonucleotide
acggggtgctgccatggaga

SEQ ID NO: 45 - DNA, 20 bp, Artificial Sequence
  Antisense Oligonucleotide
gaaaggctggtgacattgcc

SEQ ID NO: 46 - DNA, 20 bp, Artificial Sequence
  Antisense Oligonucleotide
ggatgcggttggaggacaag

SEQ ID NO: 47 - DNA, 20 bp, Artificial Sequence
  Antisense Oligonucleotide
gtcagaatcatggaggtggt

SEQ ID NO: 48 - DNA, 20 bp, Artificial Sequence
  Antisense Oligonucleotide
aggctgggcaggtgggcaaa

SEQ ID NO: 49 - DNA, 20 bp, Artificial Sequence
  Antisense Oligonucleotide
ccacttgaggttgagatgcc

SEQ ID NO: 50 - DNA, 20 bp, Artificial Sequence
  Antisense Oligonucleotide
gccaaccggcgggcagttcc

SEQ ID NO: 51 - DNA, 20 bp, Artificial Sequence
  Antisense Oligonucleotide
gggaagtgcatggggctgag

SEQ ID NO: 52 - DNA, 20 bp, Artificial Sequence
  Antisense Oligonucleotide
gctcgatggtcatgtggcag

SEQ ID NO: 53 - DNA, 20 bp, Artificial Sequence
  Antisense Oligonucleotide
cacagccaagaaggtgctgg

SEQ ID NO: 54 - DNA, 20 bp, Artificial Sequence
  Antisense Oligonucleotide
tttagctcttccagggtggg

SEQ ID NO: 55 - DNA, 20 bp, Artificial Sequence
  Antisense Oligonucleotide
agctcaggtttagctcttcc

SEQ ID NO: 56 - DNA, 20 bp, Artificial Sequence
  Antisense Oligonucleotide
tgatgttgttgtagctcagg

SEQ ID NO: 57 - DNA, 20 bp, Artificial Sequence
  Antisense Oligonucleotide
gggcagcgcaggcacagtca

SEQ ID NO: 58 - DNA, 20 bp, Artificial Sequence
  Antisense Oligonucleotide
gacagggatatgagggattt

SEQ ID NO: 59 - DNA, 20 bp, Artificial Sequence
  Antisense Oligonucleotide
ggatgttggtatggctgagg

SEQ ID NO: 60 - DNA, 20 bp, Artificial Sequence
  Antisense Oligonucleotide
gctggcagagtctagcatca

SEQ ID NO: 61 - DNA, 20 bp, Artificial Sequence
  Antisense Oligonucleotide
agggcatgcaggccggcgag

SEQ ID NO: 62 - DNA, 20 bp, Artificial Sequence
  Antisense Oligonucleotide
cgtccatgaataggaagcgc

SEQ ID NO: 63 - DNA, 20 bp, Artificial Sequence
  Antisense Oligonucleotide
gttcttgtaataacagttgc

SEQ ID NO: 64 - DNA, 20 bp, Artificial Sequence
  Antisense Oligonucleotide
tccagtgcctgcctgcaggg

SEQ ID NO: 65 - DNA, 20 bp, Artificial Sequence
  Antisense Oligonucleotide
gccacctccagtgcctgcct

SEQ ID NO: 66 - DNA, 20 bp, Artificial Sequence
  Antisense Oligonucleotide
ggagggcacccggggccacc

SEQ ID NO: 67 - DNA, 20 bp, Artificial Sequence
  Antisense Oligonucleotide
ggtgaggttgcccaggccaa

SEQ ID NO: 68 - DNA, 20 bp, Artificial Sequence
  Antisense Oligonucleotide
ttgtacttgagtgacaggtg

SEQ ID NO: 69 - DNA, 20 bp, Artificial Sequence
  Antisense Oligonucleotide
ggggcaccacagtgaggttg

SEQ ID NO: 70 - DNA, 20 bp, Artificial Sequence
  Antisense Oligonucleotide
caggctggaaggcaggttgc

SEQ ID NO: 71 - DNA, 20 bp, Artificial Sequence
  Antisense Oligonucleotide
taggacaacagcagatactc

SEQ ID NO: 72 - DNA, 20 bp, Artificial Sequence
  Antisense Oligonucleotide
ccagtttgacgatgcggttg

SEQ ID NO: 73 - DNA, 20 bp, Artificial Sequence
  Antisense Oligonucleotide
attggccaggtcctcaggcg

SEQ ID NO: 74 - DNA, 20 bp, Artificial Sequence
  Antisense Oligonucleotide
agcacacgcagggcggtcag

SEQ ID NO: 75 - DNA, 20 bp, Artificial Sequence
  Antisense Oligonucleotide
ggcaatttccgcccacatcg

SEQ ID NO: 76 - DNA, 20 bp, Artificial Sequence
  Antisense Oligonucleotide
gggagcgtggtcgcagcggc

SEQ ID NO: 77 - DNA, 20 bp, Artificial Sequence
  Antisense Oligonucleotide
gggcactccatgcaggggtt

SEQ ID NO: 78 - DNA, 20 bp, Artificial Sequence
  Antisense Oligonucleotide
gtagctgggggaagtgacga

SEQ ID NO: 79 - DNA, 20 bp, Artificial Sequence
  Antisense Oligonucleotide
gtggctgaaggtatcgggat

SEQ ID NO: 80 - DNA, 20 bp, Artificial Sequence
  Antisense Oligonucleotide
aggccttcaagacggctcag

SEQ ID NO: 81 - DNA, 20 bp, Artificial Sequence
  Antisense Oligonucleotide
gagaactgtccttcaacacc

SEQ ID NO: 82 - DNA, 20 bp, Artificial Sequence
  Antisense Oligonucleotide
actggcattcagccaggaga

SEQ ID NO: 83 - DNA, 20 bp, Artificial Sequence
  Antisense Oligonucleotide
gaaccaactggcattcagcc

SEQ ID NO: 84 - DNA, 20 bp, Artificial Sequence
  Antisense Oligonucleotide
tttcccagcccacggaacca

SEQ ID NO: 85 - DNA, 20 bp, Artificial Sequence
  Antisense Oligonucleotide
tcaggtccagcactcggagg

SEQ ID NO: 86 - DNA, 20 bp, Artificial Sequence
  Antisense Oligonucleotide
ctcactcaggtccagcactc

SEQ ID NO: 87 - DNA, 20 bp, Artificial Sequence
  Antisense Oligonucleotide
ttctcactcaggtccagcac

SEQ ID NO: 88 - DNA, 20 bp, Artificial Sequence
  Antisense Oligonucleotide
tttgtagaggaagttctcac

SEQ ID NO: 89 - DNA, 20 bp, Artificial Sequence
  Antisense Oligonucleotide
gccttggttttagtgatgca

SEQ ID NO: 90 - DNA, 20 bp, Artificial Sequence
  Antisense Oligonucleotide
gctgtgttaggccctggaag

SEQ ID NO: 91 - DNA, 20 bp, Artificial Sequence
  Antisense Oligonucleotide
ggacaggttaagcttgcgca

SEQ ID NO: 92 - DNA, 20 bp, Artificial Sequence
  Antisense Oligonucleotide
accctcttttggtaattgaa

SEQ ID NO: 93 - DNA, 20 bp, Artificial Sequence
  Antisense Oligonucleotide
gagacaggtgggcaaaggac

SEQ ID NO: 94 - DNA, 20 bp, Artificial Sequence
  Antisense Oligonucleotide
gctcccgaaggaaggggcca

SEQ ID NO: 95 - DNA, 20 bp, Artificial Sequence
  Antisense Oligonucleotide
agctccttcagggcgaccag

SEQ ID NO: 96 - DNA, 20 bp, Artificial Sequence
  Antisense Oligonucleotide
agaagatgccgtgcatgtcc

SEQ ID NO: 97 - DNA, 20 bp, Artificial Sequence
  Antisense Oligonucleotide
ggtctcatcgagtgagcgga

SEQ ID NO: 98 - DNA, 20 bp, Artificial Sequence
  Antisense Oligonucleotide
cgggccagtggccggagcgt

SEQ ID NO: 99 - DNA, 20 bp, Artificial Sequence
  Antisense Oligonucleotide
gagtctggagcatgggcagg

SEQ ID NO: 100 - DNA, 20 bp, Artificial Sequence
  Antisense Oligonucleotide
gaagttcatctgcagacgca

SEQ ID NO: 101 - DNA, 20 bp, Artificial Sequence
  Antisense Oligonucleotide
ccgagctgggcctggttgat

SEQ ID NO: 102 - DNA, 20 bp, Artificial Sequence
  Antisense Oligonucleotide
cagggaaggccctgaagatg

SEQ ID NO: 103 - DNA, 20 bp, Artificial Sequence
  Antisense Oligonucleotide
caggtccacgtagcgcaggc

SEQ ID NO: 104 - DNA, 20 bp, Artificial Sequence
  Antisense Oligonucleotide
ccgctgatgcggttgtccga

SEQ ID NO: 105 - DNA, 20 bp, Artificial Sequence
  Antisense Oligonucleotide
tggctgtcagctccgaagct

SEQ ID NO: 106 - DNA, 20 bp, Artificial Sequence
  Antisense Oligonucleotide
tccatctgcctcccccatgg

SEQ ID NO: 107 - DNA, 20 bp, Artificial Sequence
  Antisense Oligonucleotide
tgcagccagaccttctcccc

SEQ ID NO: 108 - DNA, 20 bp, Artificial Sequence
  Antisense Oligonucleotide
ccggagcaaggtccccaggc

SEQ ID NO: 109 - DNA, 20 bp, Artificial Sequence
  Antisense Oligonucleotide
gctgggagtgtccactgggg

SEQ ID NO: 110 - DNA, 20 bp, Artificial Sequence
  Antisense Oligonucleotide
ttgggcctgaagtcttcaga

SEQ ID NO: 111 - DNA, 20 bp, Artificial Sequence
  Antisense Oligonucleotide
tgaagttgagggtgctgcag

SEQ ID NO: 112 - DNA, 20 bp, Artificial Sequence
  Antisense Oligonucleotide
gttccgtgacagatccaagg

SEQ ID NO: 113 - DNA, 20 bp, Artificial Sequence
  Antisense Oligonucleotide
ggctgcacggtcaccaggtt

SEQ ID NO: 114 - DNA, 20 bp, Artificial Sequence
  Antisense Oligonucleotide
agagctgggcaaacatctcc

SEQ ID NO: 115 - DNA, 20 bp, Artificial Sequence
  Antisense Oligonucleotide
gcgcaggcactgcaggtgcg

SEQ ID NO: 116 - DNA, 20 bp, Artificial Sequence
  Antisense Oligonucleotide
gatgcagttgtggctcaggc

SEQ ID NO: 117 - DNA, 20 bp, Artificial Sequence
  Antisense Oligonucleotide
gagatgcagttgtggctcag

SEQ ID NO: 118 - DNA, 20 bp, Artificial Sequence
  Antisense Oligonucleotide
gggagccattgactgcctgc

SEQ ID NO: 119 - DNA, 20 bp, Artificial Sequence
  Antisense Oligonucleotide
accggtcagcggcaggaact

SEQ ID NO: 120 - DNA, 20 bp, Artificial Sequence
  Antisense Oligonucleotide
gacaggtctagcacctgcag

SEQ ID NO: 121 - DNA, 20 bp, Artificial Sequence
  Antisense Oligonucleotide
agaggtccagcttattgtgg

SEQ ID NO: 122 - DNA, 20 bp, Artificial Sequence
  Antisense Oligonucleotide
cgtgaatgagtgctcgtggt

SEQ ID NO: 123 - DNA, 20 bp, Artificial Sequence
  Antisense Oligonucleotide
gcctccagtcgtggtagctc

SEQ ID NO: 124 - DNA, 20 bp, Artificial Sequence
  Antisense Oligonucleotide
tgttgtagctgaggtccagg

SEQ ID NO: 125 - DNA, 20 bp, Artificial Sequence
  Antisense Oligonucleotide
ggctgttgtagctgaggtcc

SEQ ID NO: 126 - DNA, 20 bp, Artificial Sequence
  Antisense Oligonucleotide
ctgcatgccaaagggctggc

SEQ ID NO: 127 - DNA, 20 bp, Artificial Sequence
  Antisense Oligonucleotide
ctgaagttgtggcccacgcc

SEQ ID NO: 128 - DNA, 20 bp, Artificial Sequence
  Antisense Oligonucleotide
tgcgcaggtgagccacgaag

SEQ ID NO: 129 - DNA, 20 bp, Artificial Sequence
  Antisense Oligonucleotide
caggctgaggtggcgcaggg

SEQ ID NO: 130 - DNA, 20 bp, Artificial Sequence
  Antisense Oligonucleotide
ctgtggatgttgttgtgggc

SEQ ID NO: 131 - DNA, 20 bp, Artificial Sequence
  Antisense Oligonucleotide
agagctgctgggacacttgg

SEQ ID NO: 132 - DNA, 20 bp, Artificial Sequence
  Antisense Oligonucleotide
ggcccgcagcgacgtactgc

SEQ ID NO: 133 - DNA, 20 bp, Artificial Sequence
  Antisense Oligonucleotide
gcattgccgctgaagtccag

SEQ ID NO: 134 - DNA, 20 bp, Artificial Sequence
  Antisense Oligonucleotide
cggcccacatatggcccagt

SEQ ID NO: 135 - DNA, 20 bp, Artificial Sequence
  Antisense Oligonucleotide
gtgcagatagaggtctccct

SEQ ID NO: 136 - DNA, 20 bp, Artificial Sequence
  Antisense Oligonucleotide
ccgctcaggccttggaagaa

SEQ ID NO: 137 - DNA, 20 bp, Artificial Sequence
  Antisense Oligonucleotide
acaagtccagccagatcaaa

SEQ ID NO: 138 - DNA, 20 bp, Artificial Sequence
  Antisense Oligonucleotide
ggtgtgcaggcggttctggg

SEQ ID NO: 139 - DNA, 20 bp, Artificial Sequence
  Antisense Oligonucleotide
cgcagggtttggggcaggag

SEQ ID NO: 140 - DNA, 20 bp, Artificial Sequence
  Antisense Oligonucleotide
gtaggctcttggggaggttg

SEQ ID NO: 141 - DNA, 20 bp, Artificial Sequence
  Antisense Oligonucleotide
gtcacggagacgcagcacct

SEQ ID NO: 142 - DNA, 20 bp, Artificial Sequence
  Antisense Oligonucleotide
ttaaagaaggccaggtaatt

SEQ ID NO: 143 - DNA, 20 bp, Artificial Sequence
  Antisense Oligonucleotide
ggaagtggaggctccaccac

SEQ ID NO: 144 - DNA, 20 bp, Artificial Sequence
  Antisense Oligonucleotide
gaggacttccagtttgggca

SEQ ID NO: 145 - DNA, 20 bp, Artificial Sequence
  Antisense Oligonucleotide
agctggtttcctgccaggtc

SEQ ID NO: 146 - DNA, 20 bp, Artificial Sequence
  Antisense Oligonucleotide
tgccattggtcagggccttc

SEQ ID NO: 147 - DNA, 20 bp, Artificial Sequence
  Antisense Oligonucleotide
ccgggtgccagcaggcaggc

SEQ ID NO: 148 - DNA, 20 bp, Artificial Sequence
  Antisense Oligonucleotide
ctgacatccagcctccggag

SEQ ID NO: 149 - DNA, 20 bp, Artificial Sequence
  Antisense Oligonucleotide
cgaagctgatgctgttgcag

SEQ ID NO: 150 - DNA, 20 bp, Artificial Sequence
  Antisense Oligonucleotide
ggaaaagaagccgggggcca

SEQ ID NO: 151 - DNA, 20 bp, Artificial Sequence
  Antisense Oligonucleotide
tctcgcagctccttggcctt

SEQ ID NO: 152 - DNA, 20 bp, Artificial Sequence
  Antisense Oligonucleotide
cgttggcgctaaggttgagc

SEQ ID NO: 153 - DNA, 20 bp, Artificial Sequence
  Antisense Oligonucleotide
gtggtccactgtcttgaggg

SEQ ID NO: 154 - DNA, 20 bp, Artificial Sequence
  Antisense Oligonucleotide
gccaggggcccaaaccagga

SEQ ID NO: 155 - DNA, 20 bp, Artificial Sequence
  Antisense Oligonucleotide
ctagtatttgcagggcactc

SEQ ID NO: 156 - DNA, 20 bp, Artificial Sequence
  Antisense Oligonucleotide
cagagggttggcgcttacat

SEQ ID NO: 157 - DNA, 20 bp, Artificial Sequence
  Antisense Oligonucleotide
gccgccccacaggcgcagtg

SEQ ID NO: 158 - DNA, 20 bp, Artificial Sequence
  Antisense Oligonucleotide
ccagcaggaagtccataaag

SEQ ID NO: 159 - DNA, 20 bp, Artificial Sequence
  Antisense Oligonucleotide
gcacctccagcaggaagtcc

SEQ ID NO: 160 - DNA, 20 bp, Artificial Sequence
  Antisense Oligonucleotide
gggcacggcagcctgcacct

SEQ ID NO: 161 - DNA, 20 bp, Artificial Sequence
  Antisense Oligonucleotide
ttcacccggctgggcagacc

SEQ ID NO: 162 - DNA, 20 bp, Artificial Sequence
  Antisense Oligonucleotide
gctggcccggactgccacac

SEQ ID NO: 163 - DNA, 20 bp, Artificial Sequence
  Antisense Oligonucleotide
aaagatgctgaggccctgga

SEQ ID NO: 164 - DNA, 20 bp, Artificial Sequence
  Antisense Oligonucleotide
cagaggcgcaggtcctgtgc

SEQ ID NO: 165 - DNA, 20 bp, Artificial Sequence
  Antisense Oligonucleotide
aggagagggcctcatccagg

SEQ ID NO: 166 - DNA, 20 bp, Artificial Sequence
  Antisense Oligonucleotide
cgagagggcgaaacagtccc

SEQ ID NO: 167 - DNA, 20 bp, Artificial Sequence
  Antisense Oligonucleotide
ccagagccacagccagcagc

SEQ ID NO: 168 - DNA, 20 bp, Artificial Sequence
  Antisense Oligonucleotide
gcagcatgggcacacccagg

SEQ ID NO: 169 - DNA, 20 bp, Artificial Sequence
  Antisense Oligonucleotide
gtcccagccacagaggtgat

SEQ ID NO: 170 - DNA, 20 bp, Artificial Sequence
  Antisense Oligonucleotide
aggtggaagcagtaccagag

SEQ ID NO: 171 - DNA, 20 bp, Artificial Sequence
  Antisense Oligonucleotide
agggaagccaggccaggcac

SEQ ID NO: 172 - DNA, 20 bp, Artificial Sequence
  Antisense Oligonucleotide
cccactttgccgcccccgcc

SEQ ID NO: 173 - DNA, 20 bp, Artificial Sequence
  Antisense Oligonucleotide
ggcagggcatcctcatctcg

SEQ ID NO: 174 - DNA, 20 bp, Artificial Sequence
  Antisense Oligonucleotide
agaccacgaaggcatcgtag

SEQ ID NO: 175 - DNA, 20 bp, Artificial Sequence
  Antisense Oligonucleotide
tgcgctctgcgttttgtcga

SEQ ID NO: 176 - DNA, 20 bp, Artificial Sequence
  Antisense Oligonucleotide
ttgtacacccagtctgccac

SEQ ID NO: 177 - DNA, 20 bp, Artificial Sequence
  Antisense Oligonucleotide
ccagctgcccccgaagctcg

SEQ ID NO: 178 - DNA, 20 bp, Artificial Sequence
  Antisense Oligonucleotide
ccagcgcccacggcactcct

SEQ ID NO: 179 - DNA, 20 bp, Artificial Sequence
  Antisense Oligonucleotide
tccaggcacaggcggagtgc

SEQ ID NO: 180 - DNA, 20 bp, Artificial Sequence
  Antisense Oligonucleotide
caggcagccagtcgcgttcc

SEQ ID NO: 181 - DNA, 20 bp, Artificial Sequence
  Antisense Oligonucleotide
gttctcaaagagggttttgc

SEQ ID NO: 182 - DNA, 20 bp, Artificial Sequence
  Antisense Oligonucleotide
ccatagaccgaggcccacag

SEQ ID NO: 183 - DNA, 20 bp, Artificial Sequence
  Antisense Oligonucleotide
caaacagcgtcttgcggctg

SEQ ID NO: 184 - DNA, 20 bp, Artificial Sequence
  Antisense Oligonucleotide
ccggtccgtgtgggccagca

SEQ ID NO: 185 - DNA, 20 bp, Artificial Sequence
  Antisense Oligonucleotide
gcgcgcaagagaccactgac

SEQ ID NO: 186 - DNA, 20 bp, Artificial Sequence
  Antisense Oligonucleotide
gctgggccagcaggaagctg

SEQ ID NO: 187 - DNA, 20 bp, Artificial Sequence
  Antisense Oligonucleotide
gcggtcctccagcaggcgct

SEQ ID NO: 188 - DNA, 20 bp, Artificial Sequence
  Antisense Oligonucleotide
accagcaccacgacgtcctt

SEQ ID NO: 189 - DNA, 20 bp, Artificial Sequence
  Antisense Oligonucleotide
ctcaggatcaccagcaccac

SEQ ID NO: 190 - DNA, 20 bp, Artificial Sequence
  Antisense Oligonucleotide
ggccgtcagggctcaggatc

SEQ ID NO: 191 - DNA, 20 bp, Artificial Sequence
  Antisense Oligonucleotide
ccgcacatagcgggagcggc

SEQ ID NO: 192 - DNA, 20 bp, Artificial Sequence
  Antisense Oligonucleotide
cggcagaggcgctggcgcag

SEQ ID NO: 193 - DNA, 20 bp, Artificial Sequence
  Antisense Oligonucleotide
gccagaggaggacactctgg

SEQ ID NO: 194 - DNA, 20 bp, Artificial Sequence
  Antisense Oligonucleotide
ctgaccactgggctggtggg

SEQ ID NO: 195 - DNA, 20 bp, Artificial Sequence
  Antisense Oligonucleotide
agctgggcccagaagctgcg

SEQ ID NO: 196 - DNA, 20 bp, Artificial Sequence
  Antisense Oligonucleotide
ccctggtcagggccatgccc

SEQ ID NO: 197 - DNA, 20 bp, Artificial Sequence
  Antisense Oligonucleotide
gttatagaagtggtggttgt

SEQ ID NO: 198 - DNA, 20 bp, Artificial Sequence
  Antisense Oligonucleotide
ggtccctggcagaagttccg

SEQ ID NO: 199 - DNA, 20 bp, Artificial Sequence
  Antisense Oligonucleotide
ctcacggctattcggccgtg

SEQ ID NO: 200 - DNA, 20 bp, Artificial Sequence
  Antisense Oligonucleotide
ggcaccgtgcaggattccgg

SEQ ID NO: 201 - DNA, 20 bp, Artificial Sequence
  Antisense Oligonucleotide
ggtgaggtgagtgtggaggt

SEQ ID NO: 202 - DNA, 20 bp, Artificial Sequence
  Antisense Oligonucleotide
ggtcagaccaggcaggcaga

SEQ ID NO: 203 - DNA, 20 bp, Artificial Sequence
  Antisense Oligonucleotide
gagggaggcgagcaggggag

SEQ ID NO: 204 - DNA, 20 bp, Artificial Sequence
  Antisense Oligonucleotide
ctctgtgtcaggtgtggggt

SEQ ID NO: 205 - DNA, 20 bp, Artificial Sequence
  Antisense Oligonucleotide
tagcatttattgagtgcctg

SEQ ID NO: 206 - DNA, 3868 bp, Artificial Squence
  Antisense Oligonucleotide
ggaggtcttgtttccggaagatgttgcaaggctgtggtgaaggcaggtgcagcctagcctcctgctcaagctacaccctggccctccacgcatgaggccctgcagaactctggagatggtgcctacaagggcagaaaaggacaagtcggcagccgctgtcctgagggcaccagctgtggtgcaggagccaagacctgagggtggaagtgtcctcttagaatggggagtgcccagcaaggtgtacccgctactggtgctatccagaattcccatctctccctgctctctgcctgagctctgggccttagctcctccctgggcttggtagaggacaggtgtgaggccctcatgggatgtaggctgtctgagaggggagtggaaagaggaaggggtgaaggagctgtctgccatttgactatgcaaatggcctttgactcatgggaccctgtcctcctcactgggggcagggtggagtggagggggagctactaggctggtataaaaatcttacttcctctattctctgagccgctgctgcccctgtgggaagggacctcgagtgtgaagcatccttccctgtagctgctgtccagtctgcccgccagaccctctggagaagcccctgccccccagcatgggtttctgccgcagcgccctgcacccgctgtctctcctggtgcaggccatcatgctggccatgaccctggccctgggtaccttgcctgccttcctaccctgtgagctccagccccacggcctggtgaactgcaactggctgttcctgaagtctgtgccccacttctccatggcagcaccccgtggcaatgtcaccagcctttccttgtcctccaaccgcatccaccacctccatgattctgactttgcccacctgcccagcctgcggcatctcaacctcaagtggaactgcccgccggttggcctcagccccatgcacttcccctgccacatgaccatcgagcccagcaccttcttggctgtgcccaccctggaagagctaaacctgagctacaacaacatcatgactgtgcctgcgctgcccaaatccctcatatccctgtccctcagccataccaacatcctgatgctagactctgccagcctcgccggcctgcatgccctgcgcttcctattcatggacggcaactgttattacaagaacccctgcaggcaggcactggaggtggccccgggtgccctccttggcctgggcaacctcacccacctgtcactcaagtacaacaacctcactgtggtgccccgcaacctgccttccagcctggagtatctgctgttgtcctacaaccgcatcgtcaaactggcgcctgaggacctggccaatctgaccgccctgcgtgtgctcgatgtgggcggaaattgccgccgctgcgaccacgctcccaacccctgcatggagtgccctcgtcacttcccccagctacatcccgataccttcagccacctgagccgtcttgaaggcctggtgttgaaggacagttctctctcctggctgaatgccagttggttccgtgggctgggaaacctccgagtgctggacctgagtgagaacttcctctacaaatgcatcactaaaaccaaggccttccagggcctaacacagctgcgcaagcttaacctgtccttcaattaccaaaagagggtgtcctttgcccacctgtctctggccccttccttcgggagcctggtcgccctgaaggagctggacatgcacggcatcttcttccgctcactcgatgagaccacgctccggccactggcccgcctgcccatgctccagactctgcgtctgcagatgaacttcatcaaccaggcccagctcggcatcttcagggccttccctggcctgcgctacgtggacctgtcggacaaccgcatcagcggagcttcggagctgacagccaccatgggggaggcagatggaggggagaaggtctggctgcagcctggggaccttgctccggccccagtggacactcccagctctgaagacttcaggcccaactgcagcaccctcaacttcaccttggatctgtcacggaacaacctggtgaccgtgcagccggagatgtttgcccagctctcgcacctgcagtgcctgcgcctgagccacaactgcatctcgcaggcagtcaatggctcccagttcctgccgctgaccggtctgcaggtgctagacctgtcccacaataagctggacctctaccacgagcactcattcacggagctaccacgactggaggccctggacctcagctacaacagccagccctttggcatgcagggcgtgggccacaacttcagcttcgtggctcacctgcgcaccctgcgccacctcagcctggcccacaacaacatccacagccaagtgtcccagcagctctgcagtacgtcgctgcgggccctggacttcagcggcaatgcactgggccatatgtgggccgagggagacctctatctgcacttcttccaaggcctgagcggtttgatctggctggacttgtcccagaaccgcctgcacaccctcctgccccaaaccctgcgcaacctccccaagagcctacaggtgctgcgtctccgtgacaattacctggccttctttaagtggtggagcctccacttcctgcccaaactggaagtcctcgacctggcaggaaaccagctgaaggccctgaccaatggcagcctgcctgctggcacccggctccggaggctggatgtcagctgcaacagcatcagcttcgtggcccccggcttcttttccaaggccaaggagctgcgagagctcaaccttagcgccaacgccctcaagacagtggaccactcctggtttgggcccctggcgagtgccctgcaaatactagatgtaagcgccaaccctctgcactgcgcctgtggggcggcctttatggacttcctgctggaggtgcaggctgccgtgcccggtctgcccagccgggtgaagtgtggcagtccgggccagctccagggcctcagcatctttgcacaggacctgcgcctctgcctggatgaggccctctcctgggactgtttcgccctctcgctgctggctgtggctctgggcctgggtgtgcccatgctgcatcacctctgtggctgggacctctggtactgcttccacctgtgcctggcctggcttccctggcgggggcggcaaagtgggcgagatgaggatgccctgccctacgatgccttcgtggtcttcgacaaaacgcagagcgcagtggcagactgggtgtacaacgagcttcgggggcagctggaggagtgccgtgggcgctgggcactccgcctgtgcctggaggaacgcgactggctgcctggcaaaaccctctttgagaacctgtgggcctcggtctatggcagccgcaagacgctgtttgtgctggcccacacggaccgggtcagtggtctcttgcgcgccagcttcctgctggcccagcagcgcctgctggaggaccgcaaggacgtcgtggtgctggtgatcctgagccctgacggccgccgctcccgctatgtgcggctgcgccagcgcctctgccgccagagtgtcctcctctggccccaccagcccagtggtcagcgcagcttctgggcccagctgggcatggccctgaccagggacaaccaccacttctataaccggaacttctgccagggacccacggccgaatagccgtgagccggaatcctgcacggtgccacctccacactcacctcacctctgcctgcctggtctgaccctcccctgctcgcctccctcaccccacacctgacacagagcaggcactcaataaatgctaccgaaggc